(19) United States
(12) Patent Application Publication
(10) Pub. No.: US 2024/0335408 A1
(43) Pub. Date: Oct. 10, 2024

(54) Soluble Guanylate Cyclase Activators for Cancer and Fibrosis Treatment

(71) Applicant: University of Pittsburgh - of the Commonwealth System of Higher Education, Pittsburgh, PA (US)

(72) Inventors: Anthony J. Kanai, Pittsburgh, PA (US); Irina Zabbarova, Pittsburgh, PA (US); Youko Ikeda, Pittsburgh, PA (US)

(21) Appl. No.: 18/619,902

(22) Filed: Mar. 28, 2024

Related U.S. Application Data
(60) Provisional application No. 63/494,603, filed on Apr. 6, 2023.

(51) Int. Cl. A61K 31/197; A61K 31/192; A61K 31/47; A61K 31/506; A61K 31/5377; A61K 45/06

(52) U.S. Cl. CPC A61K 31/197; A61K 31/192; A61K 31/47; A61K 31/506; A61K 31/5377; A61K 45/06

(57) ABSTRACT

Provided herein are methods of treating cancer and fibrosis in a patient. The cancer may be prostate cancer. The fibrosis may be cystitis arising from a cancer therapy, such as from treatment of prostate cancer. The methods involve activation of senescent cells, such as senescent cancer cells with a soluble guanylate cyclase activator. The treatment with the soluble guanylate cyclase activator may be a primary therapy or an adjuvant therapy to a primary cancer therapy such as a chemotherapy or radiation therapy. The treatment may be administered after completion of a primary cancer therapy.

### CROSS REFERENCE TO RELATED APPLICATIONS

This application claims priority to U.S. Provisional Patent Application No. 63/494,603 filed Apr. 6, 2023, the disclosure of which is incorporated herein by reference in its entirety.

The American Cancer Society estimated that there were approximately 270,000 new cases of prostate cancer in the United States in 2022, which resulted in 34,500 deaths accounting for 10.7% of all cancer deaths in men, second only to lung cancer. Depending upon prostate-specific antigen (PSA) levels and the cancer stage determined upon biopsy, patients are either monitored or treated. Of the more severe cases that are treated, approximately one-third opted for surgical removal of their tumors, mainly by a radical prostatectomy, with the remaining patients undergoing external beam irradiation or brachytherapy, along with androgen deprivation therapy (ADT, e.g., Leuprolide) that leads to decreased testosterone production and prostate cellular proliferation. While irradiation and chemotherapy destroys the majority of cancerous cells, the surviving cells exhibit mitochondrial dysfunction and increased generation of reactive oxygen and nitrogen species (ROS and RNS) which is exacerbated by decreased mitophagy and accumulation of damaged mitochondria. Subsequent inflammation and release of TNF-α causes translocation of the transcription factor, NF-κβ, to the nucleus promoting survival pathways, cellular senescence and cytokine secretion. It is postulated that this secretory associated senescence phenotype (SASP) can promote tumor reemergence through increased expression of pro-growth Bcl-2 which inhibits pro-apoptotic BAX leading to cellular proliferation and enhance mitophagy.

Adjuvant therapies to target senescent cells present in a patient after treatment with a primary cancer therapy, such as irradiation therapy or chemotherapy, are desirable. Therapies to target senescent cells resulting in fibrosis, such as in radiation-induced cystitis in a patient after treatment with a primary cancer therapy, are desirable.

### STATEMENT REGARDING FEDERAL FUNDING

This invention was made with government support under Grant No. CA251341 awarded by the National Institutes of Health. The government has certain rights in the invention.

### SUMMARY

According to a first embodiment of the invention, a method of treating a patient having a cancer is provided. The method comprises administering to the patient after initiation of a primary cancer therapy in the patient an amount of a soluble guanylate cyclase (sGC) activator effective to promote senolytic activity and/or promote senomorphic activity (e.g., decrease senescence-associated cytokine release) in the patient.

According to a second embodiment of the invention, a method of clearing senescent cells and/or increasing senomorphic activity (e.g., decrease senescence-associated cytokine release) in a patient is provided. The method comprises administering to the patient an amount of a soluble guanylate cyclase (sGC) activator effective to clear senescent cells and/or increase senomorphic activity (e.g., decrease senescence-associated cytokine release) in the patient.

The following numbered clauses outline various aspects, embodiments, or examples of the present invention.

Clause 1. A method of treating a patient having a cancer, comprising administering to the patient after initiation of a primary cancer therapy in the patient an amount of a soluble guanylate cyclase (sGC) activator effective to promote senolytic activity and/or promote senomorphic activity (e.g., decrease senescence-associated cytokine release) in the patient.

Clause 2. The method of clause 1, wherein the patient has prostate cancer or has been treated for prostate cancer using the primary cancer therapy.

Clause 3. The method of clause 1 or 2, wherein the primary cancer therapy is radiation therapy.

Clause 4. The method of clause 1 or 2, wherein the primary cancer therapy is chemotherapy.

Clause 5. The method of any one of clauses 1-4, in which cells of the patient, such as cancer cells of the patient, exhibit a senescence-associate secretory phenotype (SASP).

Clause 6. The method of clause 5, wherein the senescent cells exhibiting a senescence-associate secretory phenotype (SASP) are cancer cells.

Clause 7. The method of clause 5, wherein the senescent cells exhibiting a senescence-associate secretory phenotype (SASP) are prostate cancer cells.

Clause 8. The method of any one of clauses 1-7, wherein the sGC activator is cinaciguat.

Clause 9. The method of any one of clauses 1-7, wherein the sGC activator is a compound of formula I:

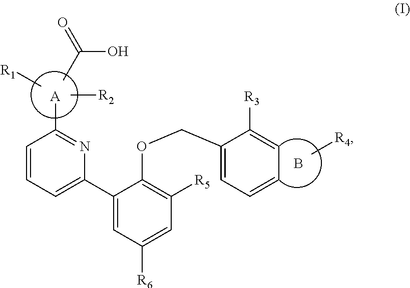

wherein:
A is a 5- or 6-membered aryl, heteroaryl or heterocyclyl group;
B is a 5-7 membered heterocyclyl group containing one nitrogen, wherein one carbon of the heterocyclyl group is optionally substituted with an oxo group, or B is a 5-membered heteroaryl group containing at least 2 nitrogens;
R₁ and R₂ are independently selected from H, C₁₋₄alkyl, C₃₋₆cycloalkyl, tetrahydropyranyl, —CF₃, —CH₂CF₃ and —CH₂CH₂CO₂H;
R₃ is selected from H and —CH₃;
R₄ is selected from H, C₁₋₆alkyl, C₃₋₇cycloalkyl, —C(O)C₁₋₆alkyl, —CH₂CF₃, —SO₂C₁₋₆alkyl, —SO₂ (CH₂)₁₋₃CO₂H, —CO₂C₁₋₄alkyl, heterocyclyl, aryl, heteroaryl,
(C₀₋₂alkyl) heterocyclyl, —(C₁₋₂alkyl) aryl and —(C₁₋₂alkyl) heteroaryl, wherein said heterocyclyl is selected from tetrahydrofuranyl, azetidinyl, pyrrolidinyl, pyranyl, tetrahydropyranyl, dioxanyl, thiomorpholinyl, 1,1-dioxo-1λ⁶-thiomorpholinyl, morpholinyl, piperidinyl, piperazinyl, and azepinyl, and said heterocyclyl, cycloalkyl, aryl and heteroaryl are optionally substituted with one to two groups independently selected from C₁₋₃alkyl, —CF₃, and halogen, or R₄ is optionally not present when B is a heteroaryl group;
R₅ is selected from H, —C₁₋₃alkyl, —OCH₃, —CF₃, —CN and Cl; and R₆ is selected from H and CH₃, provided that R₅ and R₆ are not both H;
or a salt thereof.

Clause 10. The method of any one of clauses 1-7, wherein the sGC activator is selected from one or more of Ataciguat (HMR-1766, 5-chloro-2-[(5-chlorothiophen-2-yl)sulfonylamino]-N-(4-morpholin-4-ylsulfonylphenyl)benzamide); BAY 41-2272 (5-cyclopropyl-2-[1-[(2-fluorophenyl)methyl]pyrazolo[3,4-b]pyridin-3-yl] pyrimidin-4-amine); (3S)-3-(4-chloro-3-{[(2S,3R)-2-(4-chlorophenyl)-4,4,4-trifluoro-3-methylbutanoyl]amino}phenyl)-3-cyclo-propylpropanoic acid; 5-{(4-carboxybutyl)[2-(2-{[3-chloro-4′-(trifluoromethyl) biphenyl-4-yl]methoxy}phenyl)ethyl]amino}-5,6,7,8-tetrahydroquinoline-2-carboxylic acid; and 5-{[2-(4-carboxy-phenyl)ethyl][2-(2-{[3-chloro-4′-(trifluoromethyl) biphenyl-4-yl]methoxy} phenyl)ethyl] amino}-5,6,7,8-tetrahydroquinoline-2-carboxylic acid.

Clause 11. A method of clearing senescent cells and/or increasing senomorphic activity (e.g., decrease senescence-associated cytokine release) in a patient comprising administering to the patient an amount of a soluble guanylate cyclase (sGC) activator effective to clear senescent cells and/or increase senomorphic activity (e.g., decrease senescence-associated cytokine release) in the patient.

Clause 12. The method of clause 11, wherein a primary cancer therapy is administered or has been administered to the patient prior to administering the soluble guanylate cyclase (sGC) activator to the patient.

Clause 13. The method of clause 12, wherein the patient has prostate cancer or has been treated for prostate cancer using the primary cancer therapy.

Clause 14. The method of clause 12 or 13, wherein the primary cancer therapy is radiation therapy.

Clause 15. The method of clause 12 or 13, wherein the primary cancer therapy is chemotherapy.

Clause 16. The method of any one of clauses 11-15, in which cells of the patient, such as cancer cells of the patient, exhibit a senescence-associate secretory phenotype (SASP).

Clause 17. The method of clause 16, wherein the senescent cells exhibiting a senescence-associate secretory phenotype (SASP) are cancer cells.

Clause 18. The method of clause 17, wherein the senescent cells exhibiting a senescence-associate secretory phenotype (SASP) are prostate cancer cells.

Clause 19. The method of any one of clauses 11-18, wherein the sGC activator is cinaciguat.

Clause 20. The method of any one of clauses 11-18, wherein the sGC activator is a compound of formula I:

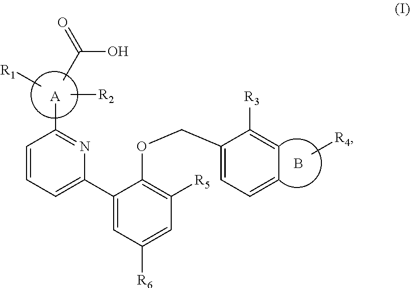

wherein:
A is a 5- or 6-membered aryl, heteroaryl or heterocyclyl group;
B is a 5-7 membered heterocyclyl group containing one nitrogen, wherein one carbon of the heterocyclyl group is optionally substituted with an oxo group, or B is a 5-membered heteroaryl group containing at least 2 nitrogens;
R₁ and R₂ are independently selected from H, C₁₋₄alkyl, C₃₋₆cycloalkyl, tetrahydropyranyl, —CF₃, —CH₂CF₃ and —CH₂CH₂CO₂H;
R₃ is selected from H and —CH₃;
R₄ is selected from H, C₁₋₆alkyl, C₃₋₇cycloalkyl, —C(O)C₁₋₆alkyl, —CH₂CF₃, —SO₂C₁₋₆alkyl, —SO₂(CH₂)₁₋₃CO₂H, —CO₂C₁₋₄alkyl, heterocyclyl, aryl, heteroaryl,
(C₀₋₂alkyl)heterocyclyl, —(C₁₋₂alkyl)aryl and —(C₁₋₂alkyl) heteroaryl, wherein said heterocyclyl is selected from tetrahydrofuranyl, azetidinyl, pyrrolidinyl, pyranyl, tetrahydropyranyl, dioxanyl, thiomorpholinyl, 1,1-dioxo-1λ⁶-thiomorpholinyl, morpholinyl, piperidinyl, piperazinyl, and azepinyl, and said heterocyclyl, cycloalkyl, aryl and heteroaryl are optionally substituted with one to two groups independently selected from C₁₋₃alkyl, —CF₃, and halogen, or R₄ is optionally not present when B is a heteroaryl group;
R₅ is selected from H, —C₁₋₃alkyl, —OCH₃, —CF₃, —CN and Cl; and R₆ is selected from H and CH₃, provided that R₅ and R₆ are not both H;
or a salt thereof.

Clause 21. The method of any one of clauses 11-18, wherein the sGC activator is selected from one or more of Ataciguat (HMR-1766, 5-chloro-2-[(5-chlorothiophen-2-yl)sulfonylamino]-N-(4-morpholin-4-ylsulfonylphenyl)benzamide); BAY 41-2272 (5-cyclopropyl-2-[1-[(2-fluorophenyl)methyl]pyrazolo[3,4-b]pyridin-3-yl] pyrimidin-4-amine); (3S)-3-(4-chloro-3-{[(2S,3R)-2-(4-chlorophenyl)-4,4,4-trifluoro-3-methylbutanoyl]amino}phenyl)-3-cyclo-propylpropanoic acid; 5-{(4-carboxybutyl) [2-(2-{[3-chloro-4′-(trifluoromethyl) biphenyl-4-yl]methoxy}phenyl)ethyl]amino}-5,6,7,8-tetrahydroquinoline-2-carboxylic acid; and 5-{[2-(4-carboxy-phenyl)ethyl][2-(2-{[3-chloro-4′-(trifluoromethyl)biphenyl-4-yl]methoxy}phenyl)ethyl]amino}-5,6,7,8-tetrahydroquinoline-2-carboxylic acid.

Clause 22. The method of any one of clauses 12-21, wherein the patient's bladder is exposed to the primary cancer therapy, and the soluble guanylate cyclase (sGC) activator is administered after the primary therapy.

Clause 23. The method of clause 22, wherein the primary cancer therapy is radiation therapy, and the patient's bladder is exposed to the radiation therapy.

Clause 24. The method of clause 22 or 23, wherein the primary cancer therapy is administered to treat prostate cancer in the patient.

Clause 25. The method of any one of clauses 22-24, wherein the patient has noninfectious cystitis after a primary cancer therapy.

Clause 26. The method of any one of clauses 22-25, wherein the soluble guanylate cyclase (sGC) activator is administered after the primary cancer therapy to treat fibrosis in the patient.

Clause 27. The method of any one of clauses 11-26, for treatment of fibrosis in the patient.

### BRIEF DESCRIPTION OF THE DRAWINGS

Drawings and data provided are exemplary and illustrative in nature.

The patent or application file contains at least one drawing executed in color. Copies of this patent or patent application publication with color drawing(s) will be provided by the Office upon request and payment of the necessary fee.

FIG. 1. Proposed mechanism for the treatment of prostate tumors using external beam radiation therapy (EBRT) and dual acting senomorphic/senolytic agent, cinaciguat. While irradiation destroys most tumor cells, surviving ones and surrounding normal cells exhibit mitochondrial dysfunction due to increased reactive oxygen and reactive nitrogen species (ROS and RNS), decreased mitophagy and mitochondrial damage. This results in decreased mitochondrial heme production, disassembly of the sGC heterodimer and increased sGCα1 subunits which increase tumor cell proliferation. The release of TNF-α causes translocation of the transcription factor, NF-κβ to the nucleus promoting survival pathways, cellular senescence and cytokine secretion. It is postulated that the secretory associated senescence phenotype (SASP) can promote tumor reemergence by promoting proliferation pathways via increased Bcl-2 and mitophagy. Currently, androgen deprivation therapy or NO· donors can inhibit this process. The SASP can be suppressed using senomorphic agents to decrease TNF-α and cytokine production to maintain the growth arrested state. Conversely, senolytic agents promote sGC heterodimer formation, apoptotic pathways and clearance of senescent cells.

FIG. 2. Regulation of sGC signaling. sGC is formed as a heterodimer of α and β subunits with a heme prosthetic group in the catalytic site on the β subunit. Cytochrome B5 reductase type 3 (CYB5R3) plays a critical role in maintaining sGC activity by reducing heme (Fe²⁺) for NO· driven cGMP production. However, oxidative stress can downregulate CYB5R3 activity or directly oxidize the heme moiety, which results in heme detachment and insensitivity of sGC to NO·. This can be circumvented by the sGC activator, cinaciguat, a heme-mimetic that does not require NO· for cGMP production. PDE5Is are structural analogs of cGMP and competitive antagonists of PDE5.

FIG. 3. Immunolocalization of sGC in normal adult mouse prostate glands. sGC β-subunit (sGCβ1, red) and smooth muscle actin (αSMA, green) in the A) lateral prostate and B) dorsal prostate gland wall. To localize NO·-induced cGMP generation, isolated male LUT was incubated in an organ bath with IBMX (1 mM) and DETA-NONOate (100 μM), fixed and cryosectioned for immunofluorescent labeling. C) Dorsal prostatic lobe with cGMP (green) and αSMA (red) labeling.

FIG. 4. The effect of single-dose and fractionated irradiation on TRAMP-C1 cell survival. A) Cells were plated at 5,000 cells/well in 96-well plates and subjected to single dose irradiation of 0 or 8 Gy. Four days following irradiation cells were treated with cinaciguat (1 μM), p62 inducer (1 μM), enzalutamide (1 μM), DETA-NONOate (100 M), 8-bromo-cGMP (100 μM) or vehicle (0.1% DMSO) for 1 week. Bioluminescence was measured before and after irradiation, and following drug treatment. Bar graph shows bioluminescence after treatment in non-irradiated and irradiated cells as percentage of readings before irradiation/treatment. B) Determination of 6 Gy as the optimal 5-day fractionated radiation dose based on cell survival.

FIG. 5. TRAMP-C1 orthotopic tumors and the effects of irradiation. A) TRAMP-C1 cells (10⁷ cells/100 μl) were injected bilaterally into the ventrolateral prostatic lobes to create the orthotopic tumor mouse model. A blue dye was added to the mixture and injected unilaterally to demonstrate that the injectate stays in the prostate. B) TRAMP-C1 tumor in a non-irradiated mouse 5 weeks after cell injection. C) Tumor following fractionated irradiation (2 Gy×5 days, starting 4 weeks post injection). D) Prostate wet weight in control, non-irradiated and irradiated tumor bearing animals.

FIG. 6. Bioluminescence imaging of prostate tumors and prostate size assessment following treatments. A) Image of colorimetrically expressed bioluminescence intensity superimposed over the mouse image. B) Prostates were excised from non-injected control, injected non-irradiated and injected irradiated (2 Gy×5 days) mice with or without treatments (P62 inducer or cinaciguat, 1 mg/kg/day for 2 weeks by ALZET pumps starting 1 week post irradiation). C) Timeline of tumor growth based on preliminary data.

FIGS. 7A and 7B. Alterations in the sGCα1, Bcl-2/BAX ratio and NF-κβ nuclear localization in prostate tumors from non-irradiated mice, and irradiated mice treated with cinaciguat or p62 inducer based on immunolabeling intensity. A)-C) sGC α1 immunolabeling in A) non-irradiated orthotopic TRAMP-C1 tumors. B) orthotopic tumor from mouse receiving fractionated irradiation followed by two weeks of cinaciguat (1 mg/kg/day) treatment. C) orthotopic tumor from mouse receiving fractionated irradiation followed by two weeks of p62 mitophagy inducer (1 mg/kg/day) treatment. D)-F) Immunolabeling for Bcl-2 of D) non-irradiated orthotopic TRAMP-C1 tumor. E) orthotopic tumor from mouse receiving fractionated irradiation and cinaciguat treatment. F) orthotopic tumor from mouse receiving fractionated irradiation and p62 mitophagy inducer treatment. G-I) Immunolabeling for NF-κβ of G) non-irradiated orthotopic TRAMP-C1 tumor. H) orthotopic tumor from mouse receiving fractionated irradiation and cinaciguat treatment. I) orthotopic tumor of mouse receiving fractionated irradiation and p62 mitophagy inducer treatment. J) Bar chart for ratio of sGCα1 positive area to total cell area. K) Bar chart of Bcl-2 positive staining versus BAX positive staining in sequential sections from each group. L) Bar chart of NF-κβ positive nuclei as percent of all nuclei in imaged area. Green fluorescence—immunolabeling, Blue—DAPI, nuclei.

FIG. 8. Mitochondrial O2 consumption recordings with a Clark oxygen electrode. Mitochondria were isolated from control, tumor bearing and tumor bearing irradiated prostates. Recordings depict four states of respiration: I, respiration with the internal substrates (addition of prostate mitochondria to air-saturated respiratory media); II, respiration stimulated by additional substrates (succinate is substrate for complex II); III, state induced by addition of high (200-300 μM) ADP; state IV when ADP is depleted by phosphorylation to ATP. A) Trace of mitochondrial respiration from a benign control mouse prostate. B) Trace of mitochondrial respiration from non-irradiated orthotopic TRAMP-C1 tumor. C) Trace of mitochondrial respiration from irradiated orthotopic TRAMP-C1 tumor. D) State III and IV respiration rates and RCR values.

FIG. 9 provides a schematic of the experimental design described in Example 1.

FIG. 10 provides a schematic of the experimental design described in Example 2.

FIG. 11 provides a schematic of the experimental design described in Example 3.

FIG. 12 Effects of cinaciguat on a mouse bladder with chronic radiation cystitis. A) Cystometry (CMG), non-irradiated mouse. B) CMG, 9 weeks following bladder irradiation (10 Gy). C) CMG, irradiation+daily gavage for 2 weeks with cinaciguat. D) Active and (E-F) passive length-tensions from irradiated+vehicle (red) vs. irradiated+cinaciguat (blue) bladder strips. G-I) Bladder cross-sections stained with Verhoeff Van Gieson solution; collagen is stained pink. J) Collagen, % area of the bladder wall, N=4. K) bladder weight in mg, control vs. irradiated (irr)>9 wks; N=5 ea.

FIG. 13 Effect of cinaciguat on bladder activity in conditional smooth muscle-specific CyB5R₃−/− mice. A). CMG and external urethral sphincter electromyogram (EUS-EMG) from a control mouse. B) CMG-EUS-EMG from a CyB5R₃−/− mouse showed reduced ICI and non-voiding contractions. C) CMG-EUS-EMG from a CyB5R₃−/− mouse following acute IP administration of cinaciguat. Active (D) and passive (E-F) tensions from control (blue) vs. CyB5R₃−/−(red) bladders strips (from, Zabbarova IV, Ikeda Y, Kanai AJ., et al. Benign prostatic hyperplasia/obstruction ameliorated using a soluble guanylate cyclase activator. J Pathol. 2022 April; 256 (4): 442-454).

FIG. 14 provides photomicrographs showing p21 staining in non-irradiated A) and irradiated mouse bladders 2 weeks B) and 9 months C) following irradiation.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include but are not limited to embodiments “consisting essentially of” and “consisting of” these stated elements or steps.

Cell senescence in general is a stress response due to different triggers including telomere shortening with aging, DNA damage due to cancer therapy, oxidative stress, or spontaneous oncogene expression resulting in prevention of cell division and resistance to cell death. Cell senescence can be a consequence of both physiological processes and pathological ones, including cancer, that are largely dependent on the microenvironment created by the senescence-associated secretory phenotype (SASP). The different paracrine activity of these SASPs determine whether there is continued decreased function as in aging, or cell survival and reemergence of hyperproliferation as in cancer. It is believed that the SASP initially prevents cell division and promotes immune clearance of damaged cells, but later creates an immunosuppressive milieu promoting tumor reemergence.

Since a SASP may cause tumor cells to reemerge, we specifically targeted these cells using a class of senotherapeutic agents-soluble guanylate cyclase activators (e.g., cinaciguat). These agents either kill senescent cells (i.e., senolytic activity) and/or inhibit their secretory characteristics (i.e., senomorphic activity). The senolytic action mainly block prosurvival/anti-apoptotic pathways, while the senomorphic action directly or indirectly attenuate the SASP by suppressing transcription factors such as NF-κβ or other pathway intermediates that contribute to the initiation and maintenance of the SASP without toxicity or cytotoxicity. Cell senescence can result from exposure of cells to ionizing radiation (external beam or brachytherapy) and chemotherapeutic agents, in which case it is referred to as therapy-induced senescence.

As used herein, the “treatment” or “treating” of a patient means administration to a patient by any suitable dosage regimen, procedure and/or administration route of a composition, device, or structure with the object of achieving a desirable clinical/medical end-point, including but not limited to, increased survival, reduction of cancer cell number, reduction of fibrosis, improvement of non-infectious (e.g., radiation-induced) cystitis symptoms, reduction of inflammation, reduction of numbers of senescent cancer cells or senescent pro-inflammatory/pro-fibrotic cell types, and/or improvement of any other suitable symptom or marker of cancer, cancer cell senescence, fibrosis, or non-infectious cystitis. An amount of any reagent or therapeutic agent, administered by any suitable route, effective to treat a patient is an amount capable of reducing cancer cell number, reduction of fibrosis, improvement of non-infectious (e.g., radiation-induced) cystitis symptoms, reduction of inflammation, reduction of numbers of senescent cancer cells or senescent pro-inflammatory/pro-fibrotic cell types, and/or improvement of any other suitable symptom or marker of cancer, cancer cell senescence, fibrosis, or non-infectious cystitis. The therapeutically-effective amount of each therapeutic may range from 1 pg per dose to 10 g per dose from 1 μg to 1 mg per dose, including any amount there between, such as, without limitation, 1 ng, 1 μg, 1 mg, 10 mg, 100 mg, 1 g, or 10 g per dose; from 1 μg/h to 1 mg/h such as 10 μg/h, 100 μg/h, 200 μg/h, or 400 μg/h; or from 0.01 μg/kg/h to 10 μg/kg/hr, such as 1 μg/kg/hr, 5 μg/kg/hr, or 10 μg/kg/hr, where a dose may be an amount administered to a patient, or perfused into an organ either in vivo or ex vivo, in a pharmaceutically-acceptable carrier. The therapeutic agent may be administered by any effective route, but in the context of treatment of cancer or fibrosis, or as an adjuvant or follow-on treatment of cancer, may be most typically delivered parenterally, e.g., as an intravenous or infusion therapy, or orally to a patient. The therapeutic agent may be administered as a single dose, continuously, at regular or irregular intervals, or in amounts and intervals as dictated by any clinical parameter of a patient.

The therapeutic agent described herein is a soluble guanylate cyclase (sGC) activator, which is administered following initiation of primary cancer treatment. “Cancer” excludes benign hyperplasia. Non-limiting examples of primary cancer treatments include chemotherapy, radiation therapy, and immunotherapy, as are broadly-known. Primary cancer therapies, such as, without limitation, chemotherapies and radiation therapy, with or without adjuvant therapies, can drive cancer cells into the senescent phenotype, such as a senescence-associate secretory phenotype (SASP). Because the cells are driven to the senescent phenotype as a result of the primary cancer therapy and are not necessarily present prior to the primary cancer therapy, the sGC activator may not be administered to patients during the primary cancer treatment, but as a follow-on or adjuvant therapy after the primary therapy. The sGC activator may be administered before the primary therapy is completed, such that the sGC activator may be administered to the patient after the primary therapy is initiated. The primary cancer therapy may continue after the sGC activator is administered. That said, in one example, the sGC activator may be administered after the primary cancer therapy is completed to prevent remission due to the presence of senescent cancer cells. In one example, the sGC activator is cinaciguat.

An adjuvant therapy refers to an additional cancer treatment given after the primary treatment to lower the risk that the cancer will come back. Adjuvant therapy may include chemotherapy, radiation therapy, hormone therapy, targeted therapy, or biological therapy. As such, treatment of cancer with sGC activator(s) as described herein is considered an adjuvant therapy.

sGC activators are a recognized class of active pharmaceutical ingredients. One example of an sGC, e.g., as tested below, is cinaciguat (also, BAY 58-2667), e.g., 4-({(4-carboxybutyl)[2-(2-{[4-(2-phenylethyl)phenyl]methoxy}phenyl)ethyl]amino}methyl) benzoic acid, and having the exemplary structure:

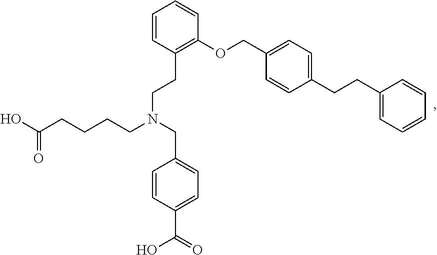 [SMILES: O=C(O)CCCCN(CCc1ccccc1OCc1ccc(CCc2ccccc2)cc1)Cc1ccc(C(=O)O)cc1]

including pharmaceutically-acceptable salts thereof. Other sGC activators include HMR 1766 and BAY 41-2272. U.S. Pat. Nos. 8,569,339 B2 and 11,331,308 B2 describe sGC activators, as well as the following, which are incorporated herein by reference only for their description of sGC activators: WO2013/157528, WO2015/056663, WO2009/123316, WO2016/001875, WO2016/001876, WO2016/001878, WO2000/02851, WO2012/122340, WO2013/025425, WO2014/039434, WO2016/014463, WO2009/068652, WO2009/071504, WO2010/015652, WO2010/015653, WO2015/033307, WO2016/042536, WO2009/032249, WO2010/099054, WO2012/058132, US2010/0216764, WO01/19776, WO01/19780, WO01/19778, WO02/070459, WO02/070460, WO02/070510, WO02/070462, WO2007/045366, WO2007/045369, WO2007/045433, WO2007/045370, WO2007/045367, WO2014/012935, WO2014/012934, WO2011/141409, WO2008/119457, WO2008/119458, WO2009/127338, WO2010/102717, WO2011/051165, WO2012/076466, WO2012/139888, WO2013/174736.

Exemplary sGC activators include, without limitation: Ataciguat (HMR-1766, 5-chloro-2-[(5-chlorothiophen-2-yl)sulfonylamino]-N-(4-morpholin-4-ylsulfonylphenyl) benzamide), BAY 41-2272 (5-cyclopropyl-2-[1-[(2-fluorophenyl)methyl]pyrazolo[3,4-b]pyridin-3-yl]pyrimidin-4-amine),(3S)-3-(4-chloro-3-{[(2S,3R)-2-(4-chlorophenyl)-4,4,4-trifluoro-3-methylbutanoyl]amino}phenyl)-3-cyclopropylpropanoic acid, 5-{(4-carboxy butyl) [2-(2-{[3-chloro-4′-(trifluoromethyl) biphenyl-4-yl]methoxy}phenyl)ethyl]amino}-5,6,7,8-tetrahydroquinoline-2-carboxylic acid, and 5-{[2-(4-carboxyphenyl)ethyl][2-(2-{[3-chloro-4′-(trifluoromethyl) biphenyl-4-yl]methoxy} phenyl)ethyl]amino}-5,6,7,8-tetrahydroquinoline-2-carboxylic acid.

The compound may have the structure of formula I (see, e.g., U.S. Pat. No. 8,569,339 B2, incorporated herein by reference for its technical disclosure):

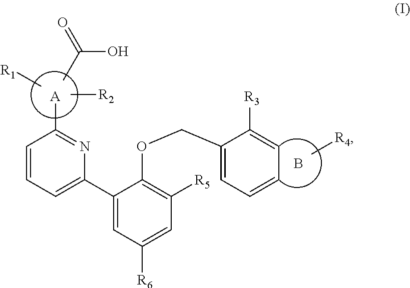

wherein:
A is a 5- or 6-membered aryl, heteroaryl or heterocyclyl group;
B is a 5-7 membered heterocyclyl group containing one nitrogen, wherein one carbon of the heterocyclyl group is optionally substituted with an oxo group, or B is a 5-membered heteroaryl group containing at least 2 nitrogens;
R₁ and R₂ are independently selected from H, C₁₋₄alkyl, C₃₋₆cycloalkyl, tetrahydropyranyl, —CF₃, —CH₂CF₃ and —CH₂CH₂CO₂H;
R₃ is selected from H and —CH₃;
R₄ is selected from H, C₁₋₆alkyl, C₃₋₇cycloalkyl, —C(O)C₁₋₆alkyl, —CH₂CF₃, —SO₂C₁₋₆alkyl, —SO₂ (CH₂)₁₋₃CO₂H, —CO₂C₁₋₄alkyl, heterocyclyl, aryl, heteroaryl, —(C₁₋₂alkyl) heterocyclyl, —(C₁₋₂alkyl) aryl and —(C₁₋₂alkyl) heteroaryl,
wherein said heterocyclyl is selected from tetrahydrofuranyl, azetidinyl, pyrrolidinyl, pyranyl, tetrahydropyranyl, dioxanyl, thiomorpholinyl, 1,1-dioxo-1λ⁶-thiomorpholinyl, morpholinyl, piperidinyl, piperazinyl, and azepinyl, and
said heterocyclyl, cycloalkyl, aryl and heteroaryl are optionally substituted with one to two groups independently selected from C₁₋₃alkyl, —CF₃, and halogen,
or R₄ is optionally not present when B is a heteroaryl group;
R₅ is selected from H, —C₁₋₃alkyl, —OCH₃, —CF₃, —CN and Cl; and R₆ is selected from
H and CH₃, provided that R₅ and R₆ are not both H;
or a salt thereof.

A “moiety” (pl. “moieties”) is a part of a chemical compound, and includes groups, such as functional groups.

Chemical moieties, groups, or substituents are described in a described using common nomenclature. For example, “Alkyl” may refer to straight, branched chain, or cyclic hydrocarbon groups including from 1 to about 20 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. “Substituted alkyl” may refer to alkyl substituted at 1 or more, e.g., 1, 2, 3, 4, 5, or 6 positions, which substituents are attached at any available atom to produce a stable compound, with substitution as described herein. “Optionally substituted alkyl” may refer to alkyl or substituted alkyl. “Halogen,” “halide,” and “halo” may refer to —F, —Cl, —Br, and/or —I. “Alkylene” and “substituted alkylene” may refer to divalent alkyl and divalent substituted alkyl, respectively, including, without limitation, ethylene (—CH₂—CH₂—). “Optionally substituted alkylene” may refer to alkylene or substituted alkylene. Alkene and alkyne, and similar designations have art-recognized meaning. “Heteroalkyl” may refer to an alkyl group with one or more carbons thereof replaced, independently, with one or more heteroatoms, such as N, O, P, and/or S. (Hetero)alkyl refers alkyl or heteroalkyl. “Heterocyclyl” may refer to cyclic heteroalkyl.

“Alkoxyl” or “alkyloxyl” refers to an —O-alkyl groups, such as methoxyl, ethoxyl, propyloxyl, etc.

“Aryl,” alone or in combination may refer to an aromatic ring system such as phenyl or naphthyl. “Aryl” also can include aromatic ring systems that are optionally fused with a cycloalkyl ring. A “substituted aryl” is an aryl that is independently substituted with one or more substituents attached at any available atom to produce a stable compound, wherein the substituents are as described herein. The substituents can be, for example, hydrocarbyl groups, alkyl groups, alkoxy groups, and halogen atoms. “Optionally substituted aryl” may refer to aryl or substituted aryl. An aryloxy group can be, for example, an oxygen atom substituted with any aryl group, such as phenoxy. An arylalkoxy group can be, for example, an oxygen atom substituted with any aralkyl group, such as benzyloxy. “Arylene” denotes divalent aryl, and “substituted arylene” may refer to divalent substituted aryl. “Optionally substituted arylene” refers to arylene or substituted arylene. A “polycyclic aryl group” and related terms, such as “polycyclic aromatic group” may refer to a group composed of at least two fused aromatic rings. “Heteroaryl” may refer to an aryl group with one or more carbons thereof replaced, independently, with one or more heteroatoms, such as N, O, P, and/or S. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl. (Hetero) aryl refers to aryl or heteroaryl.

“Cycloalkyl” may refer to monocyclic, bicyclic, tricyclic, or polycyclic, 3- to 14-membered ring systems, which are either saturated, or partially unsaturated. The cycloalkyl group may be attached via any atom. Cycloalkyl also contemplates fused rings wherein the cycloalkyl is fused to an aryl or heteroaryl ring. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein below. “Cycloalkylene” may refer to divalent cycloalkyl. The term “optionally substituted cycloalkylene” may refer to cycloalkylene that is substituted with at least 1, 2 or 3 substituents, attached at any available atom to produce a stable compound, wherein the substituents are as described herein. “Heterocycloalkyl” may refer to a cycloalkyl group with one or more carbons thereof replaced, independently, with one or more heteroatoms, such as N, O, P, and/or S. (Hetero)cycloalkyl may refer to cycloalkyl or heterocycloalkyl.

Terms combining the foregoing and other common terms refer to any suitable combination, such as arylalkenyl, arylalkynyl, heteroarylalkyl, heteroarylalkenyl, heteroarylalkynyl, heterocyclylalkyl, heterocyclylalkenyl, heterocyclylalkynyl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkylheterocyclylalkenyl, alkenylheterocyclylalkenyl, alkynylheterocyclylalkenyl, alkynylheteroarylalkynyl, alkylhererocyclylalkynyl, alkylheterocyclylalkyl, alkenylheterocyclylalkyl, alkenylheterocyclylalkynyl, alkynylheterocyclylalkyl, alkynylheterocyclylalkynyl, alkylaryl, alkenylaryl, alkynylaryl, alkylheteroaryl, alkenylheteroaryl, and alkynylhereroaryl. As an example, “arylalkylene” may refer to a divalent alkylene wherein one or more hydrogen atoms in an alkylene group is replaced by an aryl group, such as a (C₃-C₈) aryl group. Examples of (C₃-C₈) aryl-(C₁-C₆)alkylene groups include, without limitation: 1-phenylbutylene, phenyl-2-butylene, I-phenyl-2-methylpropylene, phenylmethylene, phenylpropylene, and naphthylethylene. The term “(C₃-C₈) cycloalkyl-(C₁-C₆)alkylene” may refer to a divalent alkylene wherein one or more hydrogen atoms in the C₁-C₆ alkylene group is replaced by a (C₃-C₈) cycloalkyl group. Examples of (C₃-C₈) cycloalkyl-(C₁-C₆)alkylene groups include without limitation 1-cycloproylbutylene, cycloproyl-2-butylene, cyclopentyl-1-phenyl-2-methylpropylene, cyclobutylmethylene and cyclohexylpropylene.

### EXAMPLES

Prostate cancer results in about 11% of all cancer deaths in men second only to lung cancer; radiation with androgen deprivation therapy is common, however, there is a possibility of reestablishment of tumors following treatment. Cinaciguat, a soluble guanylate cyclase (sGC) activator, eradicates senescent cells responsible for re-initiation of tumor growth in irradiated prostates. The goal of this project is to investigate the mechanisms by which cinaciguat promotes clearance of senescent cells following fractionated irradiation using an androgen-sensitive TRAMP-C1 prostate cancer mouse model delivering a tumor selective therapy to ameliorate adverse treatment side effects and prevent reemergence.

According to the American Cancer Society, there were approximately 270,000 cases of prostate cancer in the United States in 2022, resulting in 34,500 deaths or 10.7% of all cancer deaths in men second only to lung cancer. Of treated patients, about one-third opted for surgical removal of their tumors, with the remaining undergoing external beam radiation therapy (EBRT) or brachytherapy, along with androgen deprivation therapy (ADT; e.g., Leuprolide). While irradiation destroys the majority of cancerous cells, surviving ones can become senescent and resistant to treatment with increased risk of tumor reemergence. Cell senescence is a survival strategy in response to factors including DNA damage, increased reactive oxygen species (ROS) and reactive nitrogen species (RNS) and oxidative/nitrosative stress. These lead to inhibition of mitophagy and accumulation of damaged mitochondria.

Here, the efficacy of cinaciguat, a soluble guanylate cyclase (sGC) activator, is demonstrated in decreasing the Bcl-2/BAX ratio in aged mice to reverse benign prostatic hyperplasia. Preliminary data has been generated, indicating that cinaciguat also decreases Bcl-2/BAX to enhance clearance and prevent reemergence of TRAMP-C₁ orthotopic tumors in irradiated mouse prostates and in culture. The development of androgen resistance has been linked to overexpression of the sGCα1 subunit in prostate cancer suggesting sGC is an androgen regulated gene that stimulates tumor growth. There is reportedly high expression of sGCα1 but low expression of sGCβ1 in prostate tumors such that peptide knockdown of the α1 subunit is reported to be therapeutic. This may in part be due to decreased heme production by damaged mitochondria and failure of the mature sGCα1/β1 heterodimer to assemble. However, cinaciguat is a heme mimetic that promotes the formation of a heterodimer capable of catalyzing the formation of cGMP. Moreover, as cinaciguat acts only on heme-free sGC which accumulates in cells experiencing high levels of oxidative/nitrosative stress, such as following EBRT, it acts locally at the tumor site without systemic side effects in normal tissue where reduced sGC-Fe²⁺ is responsive only to nitric oxide (NO·). Cinaciguat may have the benefits of: i) decreasing the detrimental effect of sGCα1 overexpression through enhanced dimerization of sGC; ii) increases cGMP levels via heme-free sGC to decrease Bcl-2 levels and promote apoptotic clearance of senescence cells (senolytic effect) and decrease NF-κβ-mediated cytokine release to dampen the SASP (senomorphic effect); iii) acts at the tumor site to limit systemic side effects; iv) may obviate the need for ADT; and v) as cinaciguat has passed phase I/IIa clinical trials, it has the potential for rapid translation to clinical investigation. Fractionated EBRT and the androgen-sensitive, luciferase-expressing, orthotopic TRAMP-C1 prostate cancer mouse model and cultured cells may be used to characterize the therapeutic actions of cinaciguat.

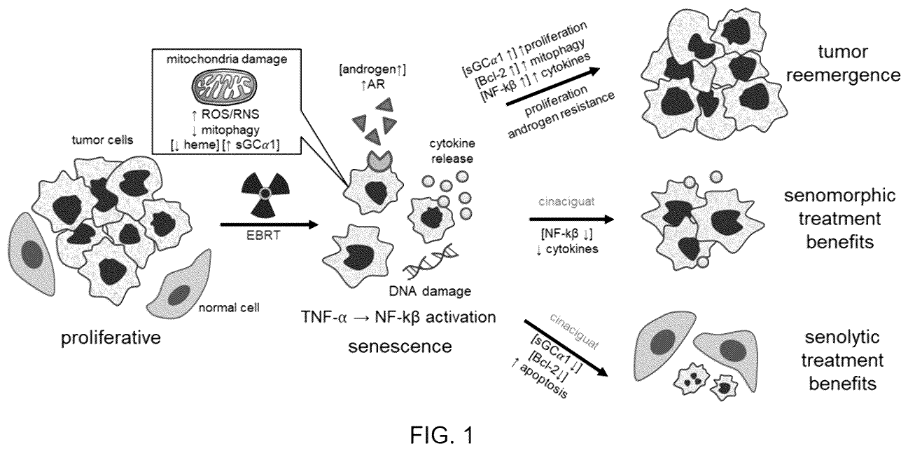

Therapy-induced cell senescence and senotherapeutic (senolytic/senomorphic) agents. Cell senescence is one of the mechanisms by which ionizing radiation (external beam or brachytherapy), chemotherapeutic agents, androgen ablation, and androgen receptor blockade exert their anti-tumor activity and is referred to as therapy-induced senescence (TIS). Cell senescence in general is a stress response due to different triggers including telomere shortening with aging, DNA damage due to cancer therapy, oxidative stress, or spontaneous oncogene expression resulting in prevention of cell division and resistance to cell death. Thus, cell senescence can be a consequence of both physiological processes as occurs during development and aging, and pathological ones including cancer that are largely dependent on the microenvironment created by the senescence-associated secretory phenotype (SASP). The different paracrine activity of these SASPs determine whether there is continued decreased function as in aging, or cell survival and reemergence of hyperproliferation as in cancer. It is believed that the SASP initially prevents cell division and promotes immune clearance of damaged cells, but later creates an immunosuppressive milieu promoting tumor re-emergence (FIG. 1).

Radiation therapy is the main strategy chosen for localized cancers in two-thirds of prostate cancer patients. Since a SASP may cause tumor cells to reemerge, specifically targeting these cells using senotherapeutics is considered to be one of the most promising strategies. These agents either kill the portion of senescent cells (i.e., senolytic) or inhibit all or part of their characteristics (i.e., senomorphic). The senolytic agents mainly block prosurvival/anti-apoptotic pathways, while the senomorphic agents directly or indirectly attenuate the SASP of senescence cells by suppressing transcription factors such as NF-κβ or other pathway intermediates that contribute to the initiation and maintenance of the SASP without excessive cytotoxicity.

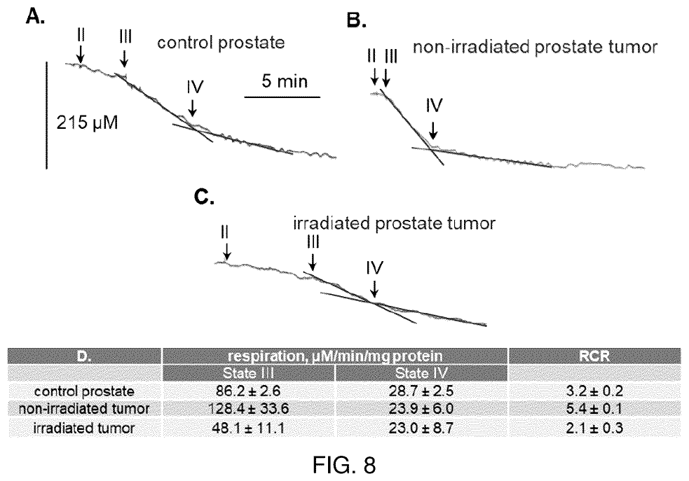

Apoptosis and mitophagy. The principal mechanisms for initiation of apoptosis are cell surface receptor (e.g., death receptor, growth factor receptors) or mitochondrial mediated pathways. In the latter, an increase in Bcl-2 proteins, or decrease in its endogenous inhibitor BAX, promotes the release of proapoptotic factors from mitochondria in response to destabilization of the outer membrane potential. Release of cytochrome c triggers activation of multiple caspase proteases leading to cleavage of intracellular proteins and ultimately cell death. Thus, Bcl-2 is one of the key oncogenic proteins in multiple malignancies. As the presence of damaged mitochondria can be detrimental, a specialized autophagy pathway called mitophagy regulates turnover of the organelles. In tumors, enhancement of mitophagy and clearance of inefficient mitochondria may increase growth. In contrast, over enhancement of mitophagy could promote cell death, thus, the contribution of mitophagy is likely dependent on the tumor's metabolic demands and oncogenic drivers. There are indications that prostate tumors have decreased mitophagy particularly in late-stage disease, potentially as a mechanism to maintain the aberrant metabolic state of resistant tumors. How mitophagy is altered following radiotherapy and whether it is involved in tumor reemergence has yet to be determined. However, tumor cells prior to EBRT have ‘super active’ mitochondria that drive proliferation as evidenced by their ultrahigh respiratory control ratios (RCRs; see FIG. 8D). This ratio decreases sharply following irradiation when cells mainly subsist on glycolysis as measured by the increased expression of β-galactosidase. RCRs may be a measure for determining the time-course of the onset of senescence when reemergence occurs and how effective cinaciguat is in clearing senescent cells.

NO·-cGMP signaling in the prostate. NO· generated from nitric oxide synthase (NOS), activates sGC by binding to the heme group on its β-subunit, inducing catalytic conversion of GTP to cGMP. The cGMP signaling cascade is controlled by a family of phosphodiesterases (PDE) that decrease the formation of cGMP and/or cAMP to play an important role in regulating cyclic nucleotide signaling pathways. Currently, 11 distinct PDE isoforms are known in various tissues, each with differing selectivity for cGMP and/or CAMP. CAMP is an important 2nd messenger for detrusor and stromal smooth muscle relaxation, regulated by PDE1 and 4 which contrasts with a smaller, albeit important role for NO·-cGMP mediated relaxation in bladder neck and urethral smooth muscle as evidenced by the positive effect of PDE5 inhibitors (PDE5Is) on LUTS. There is evidence that patients become refractory to PDE5Is with increasing age for several reasons. These include age-related degeneration of nitrergic nerves, endothelial dysfunction, and consequentially decreased NO· production; PDE5 overexpression; or inactivation/oxidation of sGC associated with inflammation and/or the production of reactive oxygen species.

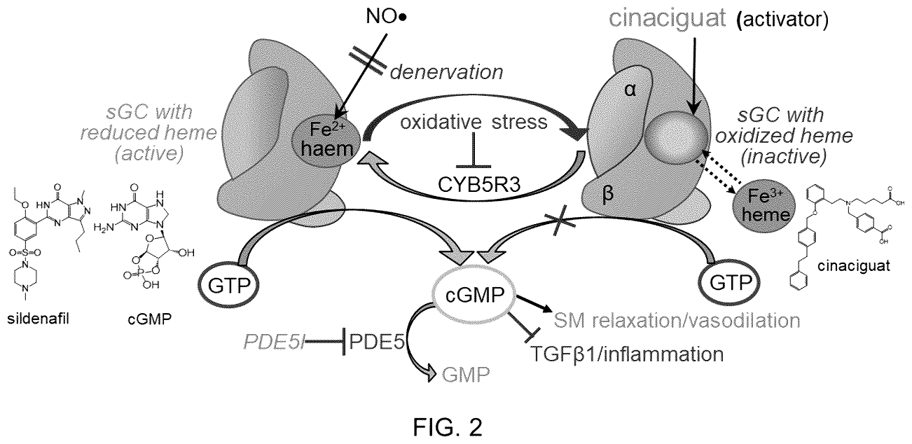

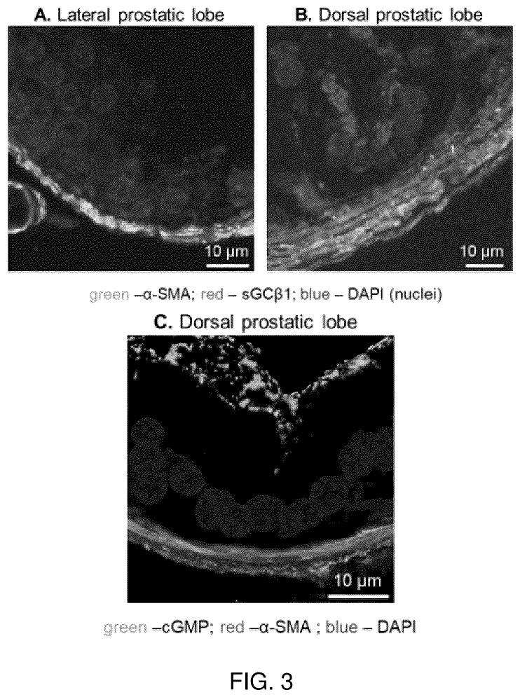

A prerequisite for cGMP production by sGC is a reduced heme (Fe²⁺) as NO· does not bind to oxidized heme (Fe³⁺), furthermore, the sGC heterodimer is destabilized when the heme moiety is lost due to oxidation. Thus, NO·-mediated cGMP production is inhibited when heme oxidation is accelerated. Cytochrome B5 reductase type 3 (CYB5R3) is a key enzyme maintaining the sGC heme in a reduced state, but its inactivation is exacerbated by inflammation or oxidative stress associated with aging and the different cancer therapies. Recent pharmacological advances have demonstrated that inactivation of sGC can be circumvented by small molecule, heme-independent sGC activators such as cinaciguat. These drugs are heme mimetics that bind to the catalytic site of sGC inducing a conformational change to directly stimulate cGMP production in the absence of NO· and when the heme is oxidized and detached (FIG. 2). Thus, sGC activators are uniquely suited for patients who may become refractory to PDE5Is or NO· donors/iNOS inducers. Moreover, the maintenance of cGMP mediated relaxation of prostatic urethra and bladder neck smooth muscle by sGC activators can address the shortcomings of current treatments such as α1-adrenoceptor antagonists. In addition to smooth muscle relaxation, sGC is highly expressed in the prostate glandular epithelium (FIG. 3).

It has recently been reported that NO· may have a modulatory role in cells with a SASP resulting in antitumor effects with the suggestion that NO· donors or iNOS inducers may be used in combination with senotherapeutic drugs. However, NO· levels should already be high in tumor cells where localized inflammation may induce iNOS. In these cells, sGC will most likely have an oxidized (Fe³⁺) or detached heme and be unresponsive to locally produced or exogenous NO· but responsive to sGC activators such as cinaciguat. Moreover, systemic administration of NO· donors and iNOS inducers may have possible unwanted systemic side effects due to toxic elevation in NO· levels, as opposed to sGC activators whose effects will be mostly localized to tumors.

Interactions of NO· and androgen receptor (AR) signaling pathways-implication for prostate cancer progression. There are reports of differential effects of NO· in prostate cancer where low levels may be growth promoting while high levels have anti-tumor effects. The latter could be in part due to direct reactions of NO· (i.e., S-nitrosylation) with proteins to induce cytotoxic effects or modulation of AR binding to its DNA target sites. Furthermore, a phase II clinical trial showed a significantly prolonged PSA doubling time in prostate cancer patients supplemented with glyceryl trinitrate patches following radiation or surgical treatments. However, glyceryl trinitrate (i.e., nitroglycerin) must first be converted by aldehyde dehydrogenase-2 in the mitochondria to nitrite (NO₂₋) which is then reduced to NO· in the cytosol. Accordingly, as damaged mitochondria and cellular senescence ensue, glyceryl trinitrate patches are less likely to slow tumor development. More recently the downstream effector of cGMP, PKG1-β, has been shown to interact with androgen response elements to regulate transcription of multiple genes. Specifically, PKG1-β induces nuclear translocation of p44 to induce growth arrest of prostate cancer cells. However, the inflammation and oxidative stress in the tumor environment will most likely lead to a shift from reduced to oxidized sGC-Fe³⁺ and decreased cGMP/PKG levels. Taken together, these support the potential for targeting cGMP signaling in prostate cancer treatment to improve the therapeutic outcomes.

The development of androgen resistance and tumor reemergence has been connected to the overexpression of the sGCα1 subunit in prostate cancer cells, which has been identified as an androgen regulated gene that stimulates tumor growth. There is reportedly high expression of sGCα1 but low expression of sGCβ1 in prostate cancer and it has been suggested that peptide knockdown of this α1 subunit may be therapeutic. NO· has been demonstrated to rapidly mobilize cellular heme to trigger subunit assembly and sGC enzyme maturation. This process requires cellular heme from mitochondria, the chaperone Hsp90 and various redox enzymes. It could be posited that in prostate cancer cells, sGCα1 does not readily form a heterodimer with the β1 subunit as elevated expression of Hsp90 may sequester the β1 subunit accounting for its low levels. Alternatively, the inflammatory conditions in prostate cancer can cause upregulation of iNOS where constitutively high levels of NO· can directly inhibit heme production and transport and thus formation of the NO·-responsive sGC. However, a heme mimetic such as cinaciguat, can promote formation of a mature sGC heterodimer, composed of either an α1 or α2 with a β1 subunit, capable of catalyzing the formation of cGMP.

Cinaciguat may have the combined benefit of i) decreasing the detrimental effect of sGCα1 overexpression through enhanced dimerization of sGC and ii) increasing cGMP levels in the absence of heme or under chronic oxidative stress to decrease Bcl-2 levels to promote apoptotic clearance of senescence cells (senolytic effect) and decrease NF-κβ induced cytokine release to dampen SASP (senomorphic effect) (FIG. 1). As cinaciguat is a heme-independent sGC activator, it acts only on heme-free sGC which accumulates in cells experiencing high levels of oxidative stress, such as following fractionated EBRT or advanced age. Thus, it is believed that modulation of the cGMP pathway with cinaciguat offers an innovative mechanism to selectively target radiation-induced senescence tumor cells and cytokine mediated side effects to reduce the potential for redevelopment of malignancies; a novel multiple step therapy (EBRT/↓sGCα1/↓Bcl-2/↓NF-κβ/↑ apoptosis of senescence cells) for preventing the reemergence of prostate tumors while limiting side effects with an increased safety profile due to selective targeting of heme-free sGC in tumor tissue, perhaps without the need for concurrent ADT.

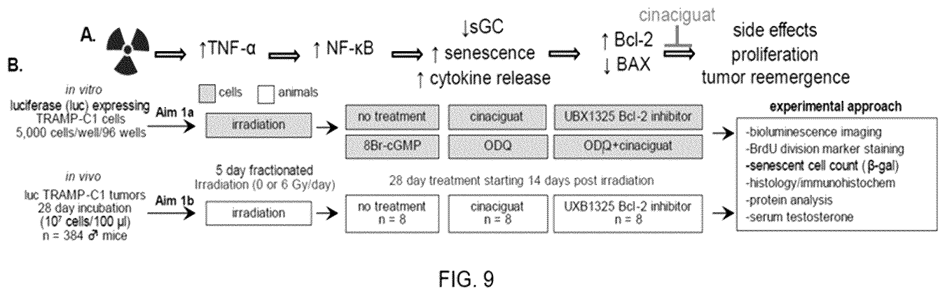

The efficacy of cinaciguat was initially examined for a cardiovascular disease but did not live up to expectations as it induced hypotension when given via i.v. drip. The impact was assessed of once-a-day oral cinaciguat treatment (10 mg/kg), which was used to ameliorate prostatic hyperplasia, with only transient effects on blood pressure and heart rate. Preliminary data also suggest that cinaciguat may be effective in treating orthotopic senescence TRAMP-C1 tumor cells, without adversely affecting blood pressure, when infused post irradiation at the lower concentrations of 0.1-1 mg/kg/day for 28 days via osmotic pumps (see FIG. 9 and Table 1).

TABLE 1

| | | | In vitro | | In vivo |
|---|---|---|---|---|---|
| Drugs | Target | Solute | conc. | | conc. |
| Cinaciguat | sGC heme | DMSO | 0.1-1 | μM | 0.1-1 mg/ |
| | binding site | | | | kg/day |
| 8-bromo-cGMP | PKG, non- | H₂O | 10-100 | μM | — |
| | hydrolysable | | | | |
| | cGMP | | | | |
| DETA-NoNoate | NO• donor | DMSO | 10-100 | μM | 10-50 mg/ |
| | | | | | kg/day |
| ODQ | sGC, heme | DMSO | 10-100 | μM | 1-5 mg/ |
| | displacer | | | | kg/day |
| UBX1325 | Bcl-2 inhibitor | DMSO | 1-10 | μM | 0.1-1 mg/ |
| | | | | | kg/day |
| P62 mitophagy | p62 | DMSO | 1-10 | μM | 0.1-1 mg/ |
| inducer | | | | | kg/day |
| Enzalutamide | AR blocker | DMSO | 10-100 | nM | 10-50 mg/ |
| | | | | | kg/day |
| Leuprolide | Gonadotropin- | DMSO | — | | 0.1-1 mg/ |
| | releasing | | | | kg/day |
| | hormone | | | | |
| | receptor agonist | | | | |

### Results:

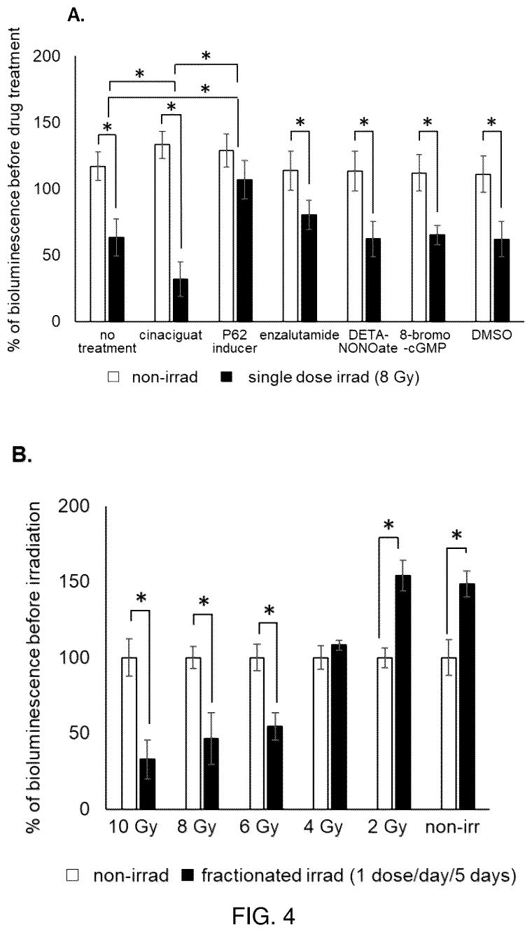

Effect of drugs targeting NO·-cGMP pathway, mitophagy and AR signaling on TRAMP-C1 survival after single dose irradiation and determination of optimal fractionated irradiation protocol. To assess whether promoting NO·-cGMP signaling, induction of mitophagy or AR inhibition would enhance the effect of radiation treatment, the effect of these different drugs on cultured luciferase (luc) expressing TRAMP-C1 cells was examined in a 96-well plate format. Bioluminescence was assessed before irradiation after which the cells were exposed to a single 8 Gy radiation dose. Four days after irradiation cells were treated with the different drugs for a further 7 days. At the conclusion of treatment, cinaciguat treated cells had the lowest survival (31±13% of bioluminescence before irradiation) while NO· donor (62±13%, DETA-NoNoate), the cGMP analog (65±7%, 8-bromo-cGMP) and AR antagonist (80±11%) showed no significant difference to vehicle or no drug (63±13% and 61±13%) (FIG. 4 (A)). These data indicate cinaciguat promotes cell death after irradiation in a manner independent of cGMP and androgen receptor signaling. Interestingly, the p62 mitophagy inducer increased survival (107±15%) indicating enhanced clearance of mitochondria is associated with recovery and growth of TRAMPC1 cells. To determine the optimal fractionated irradiation dosing protocol, fractionated irradiation protocols were examined in cultured cells over a 5-day period. These experiments showed effective killing at 6 Gy after the final radiation dose (54±9%), with no further change at higher dosages (FIG. 4 (B)). Therefore, 6 Gy per day for 5 days can be used as a dosing protocol.

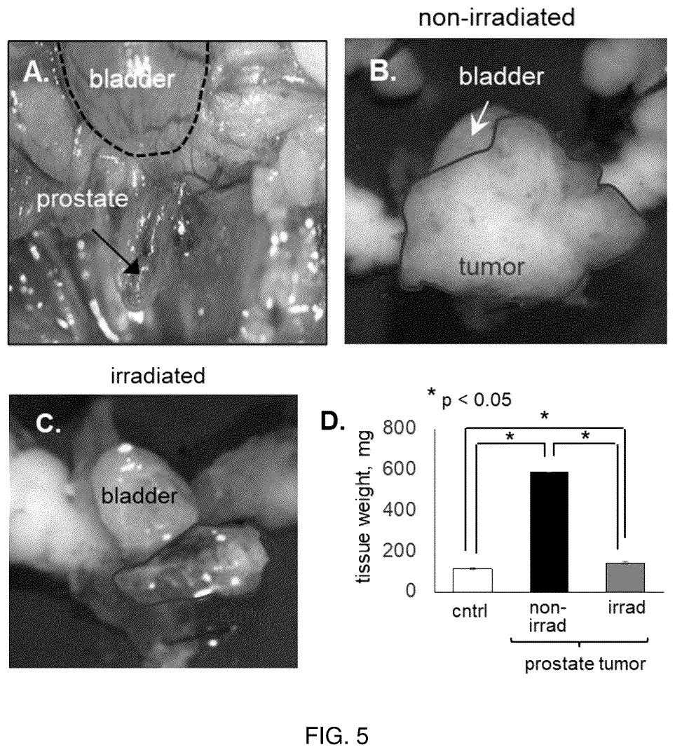

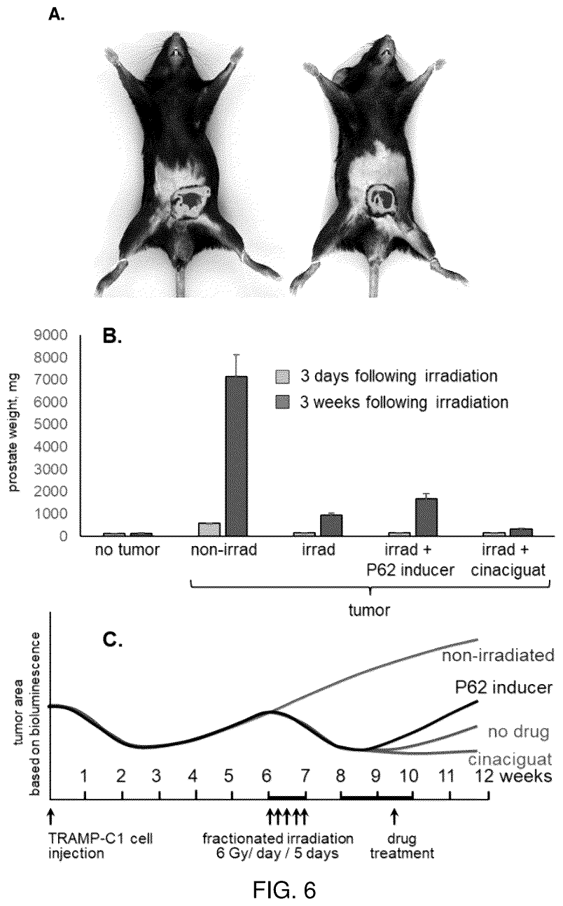

Development of orthotopic TRAMP-C1 tumors in C57BI/6 mice and the effect of fractionated irradiation. Orthotopic tumors were established by injection of TRAMP-C1 cells into the ventral prostate lobes bilaterally, which allows distribution of cells in the ventrolateral lobes (FIG. 5 (A)). Five weeks after injection, a solid mass tumor develops which engulfs the ventrolateral lobes and part of the urinary bladder (FIG. 5 (B)). Tumor bearing mice that were subjected to 2 Gy fractionated irradiation showed significant shrinkage (FIG. 5 (C and D)). These were correlated with bioluminescence imaging from mice injected with luc-TRAMP-C1 cells (FIG. 6 (A)). To correlate the cell assay findings in vivo, tumor bearing mice were subjected to fractionated irradiation (2 Gy fractions for 5 consecutive days). After one week, mice were implanted with osmotic pumps containing vehicle, cinaciguat or p62 mitophagy inducer (1 mg/kg/day for 14 days). At the conclusion of the treatment, tumor weights were evaluated. Without any intervention (drug or irradiation), there was a significant increase in tumor mass evaluated as total prostate tissue weight (FIG. 6 (B)). Irradiation alone significantly reduced prostate weight compared to non-irradiated. The p62 mitophagy inducer resulted in higher prostate weight indicating more surviving tumor tissue while cinaciguat caused a marked decrease compared to irradiation alone. These data correlate with in vitro data that cinaciguat stimulates tumor cell apoptosis after irradiation while increasing mitophagy can promote recovery of tumor cells and subsequent regrowth. Based on this data, a proposed time course for orthotopic tumor experiments is depicted in FIG. 6 (C).

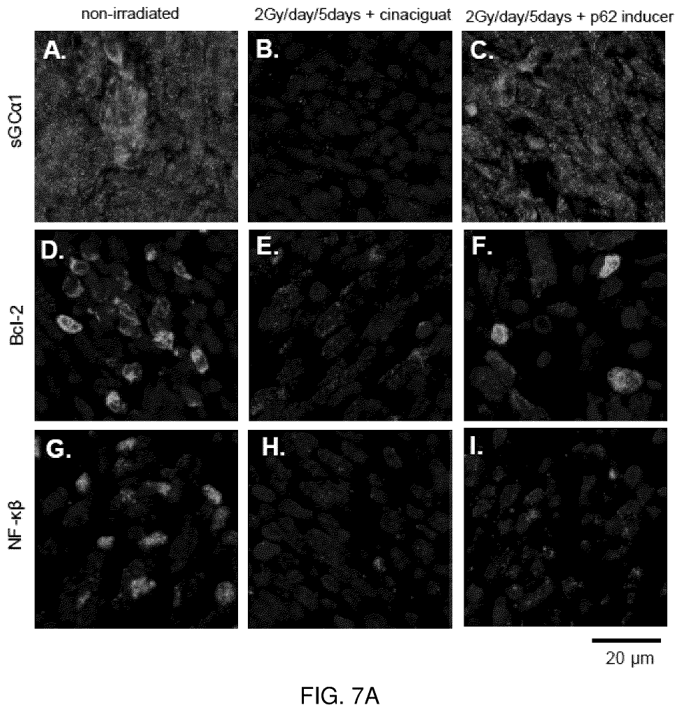

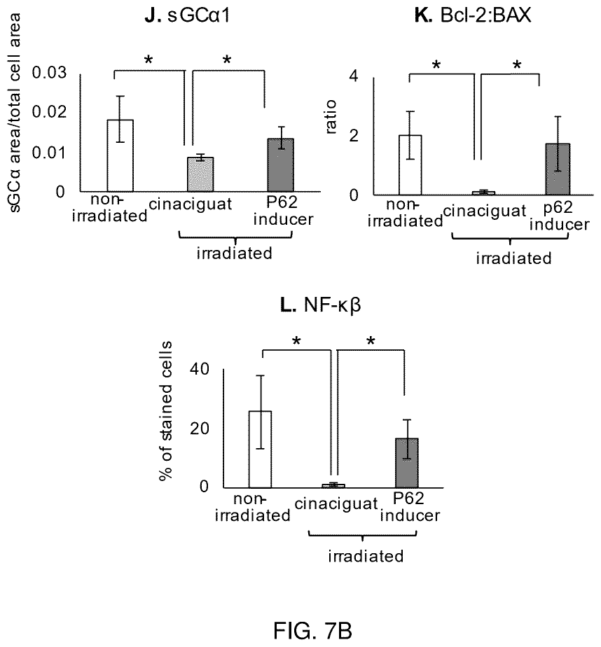

TRAMP-C1 orthotopic tumors exhibit increased sGCα1, Bcl-2 and NF-κβ expression which are reduced by cinaciguat. Immunolocalization of sGCα1, Bcl-2/BAX and NF-κβ performed in orthotopic TRAMP-C1 tumor tissue sections showed elevated labeling for these markers in non-irradiated tumors (FIGS. 7A and 7B (A, D, G, J, K, L)). These proteins are associated with increased cell growth and inflammatory signaling pathways which promote tumor progression. In the mice treated with irradiation plus cinaciguat, all three markers were reduced (FIGS. 7A and 7B (B, E, H, K)). The p62 mitophagy inducer treated mice that showed elevation of the markers (FIGS. 7A and 7B (C, F, I, L)) which could account for the increased cell survival and tumor mass following irradiation seen in vitro and in vivo, respectively. Together, the data suggest cinaciguat reduces tumor survival after irradiation by decreasing cell growth by reducing sGCα1 and Bcl-2/BAX ratio and inflammatory responses via NF-κβ inhibition.

Radiation treatment affects prostate mitochondrial respiratory control ratios (RCRs). The effect of ionizing radiation on mitochondrial RCRs was evaluated from mitochondria isolated from orthotopic TRAMP-C1 tumors (FIG. 8). Benign prostate tissue showed a mitochondrial RCR of 3.2±0.2 (ratio of respiration rate from state III and IV, FIG. 8A) which is considered a close to ideal value (between 3 to 4). Orthotopic tumors showed a significantly elevated RCR compared to benign tissues (FIG. 8 (B)). In orthotopic tumors subjected to fractionated irradiation, the isolated mitochondria showed a much-reduced RCR (FIG. 8 (C)). The decrease in mitochondrial function is believed to be one of the driving forces for radiation induced senescence and could explain the ability of p62 mitophagy inducer (clear damaged mitochondria) to promote tumor survival/growth after irradiation.

The American Cancer Society estimated that there were approximately 270,000 new cases of prostate cancer in the United States in 2022, which resulted in 34,500 deaths accounting for 10.7% of all cancer deaths in men, second only to lung cancer. Depending upon prostate-specific antigen (PSA) levels and the cancer stage determined upon biopsy, patients are either monitored or treated. Of the more severe cases that are treated, approximately one-third opted for surgical removal of their tumors, mainly by a radical prostatectomy, with the remaining patients undergoing external beam irradiation or brachytherapy, along with androgen deprivation therapy (ADT, e.g., Leuprolide) that leads to decreased testosterone production and prostate cellular proliferation. While irradiation destroys the majority of cancerous cells, the surviving cells exhibit mitochondrial dysfunction and increased generation of reactive oxygen and nitrogen species (ROS and RNS) which is exacerbated by decreased mitophagy and accumulation of damaged mitochondria. Subsequent inflammation and release of TNF-α causes translocation of the transcription factor, NF-κβ, to the nucleus promoting survival pathways, cellular senescence and cytokine secretion. It is postulated that this secretory associated senescence phenotype (SASP) can promote tumor reemergence through increased expression of pro-growth Bcl-2 which inhibits pro-apoptotic BAX leading to cellular proliferation and enhance mitophagy.

The efficacy of cinaciguat, a soluble guanylate cyclase (sGC) activator, has been previously demonstrated in decreasing the Bcl-2/BAX ratio in aged mice to reverse benign prostatic hyperplasia with preliminary data that it acts similarly with irradiated mouse prostate tumors. There are a considerable number of senotherapeutic agents in discovery, preclinical and clinical trials, the actions of many being diminution of Bcl-2 inhibition of BAX, promoting the apoptosis/clearance of senescent cells but risk severe systemic side effects. Cinaciguat, however is unique in that it is an sGC heme mimetic that selectively activates oxidized/heme-free sGC which predominates in injured/irradiated tissues to increase cGMP/PKG levels. Cinaciguat decreases NF-κβ activation to limit cytokine induced inflammatory side effects, and Bcl-2 expression to promote apoptosis and clearance of senescent cells; without affecting normal tissue where reduced sGC-Fe²⁺ is responsive to nitric oxide (NO·) but not cinaciguat. As CGMP/PKG have been demonstrated to inhibit androgen receptors in the prostate in a dose dependent manner, cinaciguat may also obviate the need for leuprolide-like agents and their accompanying side effects in prostate cancers after radiotherapy. Moreover, NO· is reported to exhibit a two-concentration effect, where low levels promote the development of prostate cancer, while high levels inhibit its growth. Thus, NO· releasing agents and inducible nitric oxide (iNOS) inducers have been tested in various cancers including castration-resistant prostate tumors. However, NO· levels should be high in tumor cells where localized inflammation will have increased iNOS expression but sGC will most likely have an oxidized (Fe³⁺) or detached heme and be unresponsive to locally produced or exogenous NO· but will be responsive to cinaciguat. Administration of NO· donors and iNOS inducers may also cause unwanted systemic side effects due to toxic elevation in NO· levels, as opposed to sGC activators whose effects will be mostly localized to tumors.

The effect of cinaciguat treatment may be evaluated following fractionated irradiation of luciferase-expressing TRAMP-C1 cell in cultures and orthotopic/prostate tumors, for non-invasive monitoring of bioluminescence in cells with and without androgen receptor blockade, and in young adult C57BI/6 mice with and without androgen ablation to decrease cytokine secretions and their accompanying side effects, and to induce apoptosis and removal of radiation-induced senescent cells in the prostate.

Accordingly, routes of inquiry may include, use or fractionated irradiation of cells and mice to investigate the dual actions of senotherapeutic cinaciguat: senomorphic action-inhibition of cytokine release and accompanying side effects; and senolytic action
apoptotic clearance of senescent cell and prevention of tumor reemergence. As cinaciguat has passed phase I and IIa clinical trials, this dual acting, tumor site selective, senotherapeutic agent has considerable potential for rapid translation to large animal toxicology studies and then clinical investigation, for example as the following:
   1) Determine the time course for development of cell senescence and reemergence, and the therapeutic benefit of cinaciguat following fractionated irradiation of bioluminescent TRAMP-C1 cells and orthotopic prostate tumors.
   2) Determine if mitochondrial dysfunction contributes to the development of radiation-induced cell senescence and tumor reemergence.
   3) Determine the effects of a nitric oxide donor versus androgen signaling inhibition following radiation treatment on cell senescence and tumor reemergence.

### Example 1

Monitor the time course for development of cell senescence and tumor reemergence and evaluate the therapeutic benefit of cinaciguat following fractionated irradiation of bioluminescent TRAMP-C1 cells and orthotopic prostate tumors. The development of cellular senescence is one of the mechanisms by which radiation and other cancer therapies inhibit tumor growth but is also the basis for tumor reemergence. In tumors, senescent cells can promote resistance to therapy and relapse, while in normal tissue they can contribute to therapy-induced side effects. Senescence results from inhibition of mitophagy, the failure to clear damaged mitochondria, and involves the inhibition of mitochondrial function, cell growth arrest, but also development of SASP cytokine mediated side effects. Thus, it is important to determine the fractionated EBRT dose that is most effective in killing tumor cells and the optimal therapeutic window to begin senolytic/senomorphic therapy to allow the development of senescence tumor cells but to prevent reemergence while limiting adverse side effects. Preliminary studies quantifying bioluminescence from luciferase expressing TRAMP-C1 cells receiving 2, 4, 6, 8, and 10 Gy fractionated irradiation daily for five days demonstrated that 6 Gy was the optimal dose (FIG. 4 (B)). EBRT was tested as low as 2 Gy and as many as 10 fractions but found these to be less effective (not shown). This is in agreement with the AUA/ASTRO guidelines recommending dose-escalated hypofractionated EBRT along with a risk-appropriate course of ADT.

The experimental design for this Example is shown in FIG. 9 where 8-Bromo-cGMP, a nonhydrolyzable analog that increases cGMP levels; UBX1325, an inhibitor of Bcl-2 that decreases Bcl-2/BAX and enhances apoptosis; and ODQ which displaces heme from sGC decreasing the enzyme's responsiveness to NO· while increasing its reactivity with cinaciguat to convert GTP to cGMP are tested. The different targets, solutes and concentrations to be tested are listed in Table 1 and the various protein targets and their actions in Table 2.

TABLE 2
Protein targets and their actions

| | | | |
|---|---|---|---|
| SGCα1 | sGC regulatory subunit | pAMPK | ROS-activated PINK1 |
| | | | kinase |
| SGCβ1 | sGC catalytic subunit | Parkin | Target of PINK1, |
| | | | mitochondrial |
| | | | ubiquitination inducer |
| Bcl-2 | Proliferation Marker | P62 | Chaperone for |
| | | | ubiquitinylated |
| | | | mitochondria to |
| | | | mitophagy pathway |
| BAX | Apoptosis Marker | HIF-1α | Hypoxia induced factor |
| | | | 1α, upregulates beclin-1 |
| NF-κβ | Stress response | Beclin-1 | parkin-independent |
| | transcription factor | | mitophagy inducer |
| Cleaved | Active apoptosis | AR | Androgen receptor |
| caspase- | markers | | |
| 3/9 | | | |
| Ki67 | Active proliferation | HSP90 | Heat-shock protein 90, |
| | marker | | chaperone for SGCβ1 |
| | | | subunit |
| P21/p16 | Cell senescence | T1/T2 | Testosterone, endogenous |
| | markers | | agonist for AR |

Determine the temporal and dose-dependent changes in TRAMP-C1 cell senescence following ionizing radiation exposure and assess senomorphic/senolytic effects of cGMP pathway activation. Preliminary studies in luciferase expressing TRAMP-C1 cells receiving single dose 8 Gy EBRT (see FIG. 4 (A)) show pretreatment with 8-bromo-cGMP or NO· donor DETA-NONOate did not enhance irradiation-induced cell death. Conversely, the androgen receptor blocker, enzalutamide, protected the cells but not to the same extent as the p62 mitophagy inducer which showed growth despite irradiation. Cinaciguat was most effective in promoting irradiation-induced tumor cell death through a cGMP-independent pathway. These results lead to the postulation that cinaciguat may be working by promoting sGC heterodimer formation with the sGCα1 subunit thereby decreasing its overexpression and limiting its proposed castration-resistance and tumor proliferation effects. Therefore, the cGMP-dependent and independent effects of cinaciguat on irradiation induced senescence and development of SASP are investigated.

The mechanisms affected by cinaciguat to limit SASP development and promote apoptosis may be initially examined in 96-well luc-TRAMP-C1 cultures. Cultures may be subjected to fractionated irradiation (6 Gy/day/5 days), then treated with of cinaciguat (1 μM), the Bcl-2 inhibitor UBX1325 (0.1-1 μM) or the non-hydrolysable cGMP analog, 8-bromo-cGMP (10-100 UM) starting four days after final radiation treatment and monitored for one week by bioluminescence. The pro-oxidative effect of radiation may be mimicked with the sGC heme displacer, ODQ (50 UM). ODQ treatment may also be combined with cinaciguat to demonstrate the selectivity of the sGC activator to the oxidized/heme-free sGC. These experiments should support preliminary findings that cinaciguat has a cGMP-independent action to promote apoptosis rather than activation of cell senescence. To show the effect of cinaciguat on sGC subunit expression and assembly, a modified western blot protocol may be used to simultaneously quantify sGC dimer and free subunits which may be obtained from cells cultured in 100 mm² dishes. In separate experiments, cell proliferation may be evaluated by BrdU incorporation assay, while cell senescence may be determined by quantifying activity of β-galactosidase at the end of treatment protocols using commercially available assays.

Evaluate progression and shrinkage after fractionated irradiation of orthotopic luciferase expressing TRAMPC1 tumors and follow-on treatment with cinaciguat or UXB1325 Bcl-2 inhibitor. There is a considerable number of senotherapeutic agents in discovery, preclinical and clinical trials, the actions of many being diminution of Bcl-2 mediated inhibition of BAX, promoting apoptosis of senescent cells. The effects of Bcl-2 inhibitor, UXB1325, to support that cinaciguat acts on the Bcl-2 pathways may be tested. The dosage ranges for in vivo studies are shown in Table 1.

Studies by others using orthotopic tumors in mice with 15 Gy single fraction EBRT suggested the optimal time to begin senescence drug therapy is 12-14 days which correlates with these findings. In these studies, bioluminescence of orthotopic TRAMP-C1 cells, injected into the dorsal and lateral prostatic lobes (10⁷ cells total), is immediately detectable. However, as these are immunocompetent mice, there is an initial decrease in cell numbers followed by establishment of the tumor by 5-6 weeks after which it continues to grow (FIG. 6 (C)). Accordingly, six weeks post injection of C57BI/6 adult mice (8-9 weeks old), animals may receive 5 consecutive days of fractionated EBRT (6 Gy/day). With this radiation regimen, tumor shrinkage is visible within 3 days post irradiation (FIG. 6 (B)) and ebbs between 7 to 14 days after the final radiation fraction. It is during this two week stationary period, senescent cells are believed to be present and senotherapy should commence. To pinpoint the optimal therapeutic window, when the tumor is smallest, bioluminescence changes may be quantified twice weekly.

After irradiation, the prostate tissues may be removed and used for mitochondrial RCRs (see FIG. 8), histology/immunohistochemistry and for the other experimental approached outlined in FIG. 9. This includes β-galactosidase activity and immunolabeling for Bcl-2, BAX, Ki67 (proliferation marker), p21/p16 (senescence markers) in frozen tissue sections. Paraffin-embedded sections of TRAMP-C1 prostate tumors stained with hematoxylin and eosin may also be compared with de-identified human tumor tissue samples before and after EBRT, to determine if similar pathological features occur to tumor cells following radiation exposure. Protein and RT-PCR may be utilized to verify molecular changes to Bcl-2, BAX, sGC subunits, SASP cytokines (e.g., IL-β1, IL-6) and p21/p16. During treatment, two-hour urine spot tests and tail cuff BP and HR photoplethysmography may be performed to monitor potential physiological changes due to irradiation or tumor burden. Separate cohorts of mice may receive either cinaciguat (1 mg/kg/day) or UXB1325 (0.1 mg/kg/day) via osmotic pumps two weeks after the final irradiation dose and may be monitored for an additional 28 days to determine if there is tumor reemergence. At the end of treatment, mice may be sacrificed for tissue collection and analyzed using molecular techniques outlined above.

Of note, a component of cell senescence is the increased expression of β-galactosidase as a marker as they subsist on glycolysis. However, it has been reported that this marker may lack specificity. If this proves to be correct, and it is not believed to be the case, other markers such as p16, p21 or p53 may be employed.

### Example 2

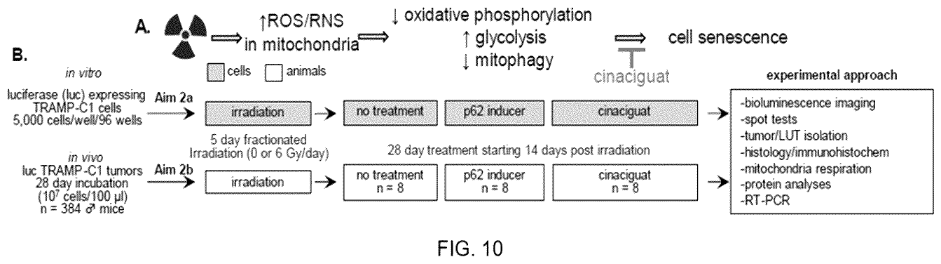

Determine if mitochondrial dysfunction contributes to the development of radiation-induced cell senescence and tumor reemergence. High cellular metabolic activity is crucial for maintaining the high growth rate of tumors. This is in part due to enhanced mitochondrial biogenesis and turnover (mitophagy) that facilitate efficient energy production and biosynthetic activities. Conversely, cell senescence is associated with defective mitophagy and decreased oxidative phosphorylation which accounts for the increased β-galactosidase activity in senescent cells that have come to rely on anaerobic glycolysis. It is postulated that radiotherapy induces senescence of tumor and normal cells within the prostate. Initially, the senescent state prevents tumor progression. However, development of a pro-inflammatory tumor microenvironment reactivates growth potentially through effects on mitophagy pathways that restore tumor cell metabolic function. Preliminary data indicate orthotopic TRAMP-C1 tumors have higher RCRs than benign prostate tissue and that fractionated irradiation decreases these values (FIG. 8). It is postulated that irradiation induces tumor senescence by reducing mitophagy, thus reducing the number of efficient mitochondria, energy production and cell growth. Alterations to mitochondrial activity of TRAMP-C1 cells following fractionated irradiation may be examined by RCR measurements and the changes to mitophagy pathway intermediates in cultured TRAMP-C1 cells and orthotopic tumors. Further, the effects of cinaciguat and the p62 mitophagy inducer treatment on mitochondrial respiration may be evaluated from orthotopic tumors (FIG. 10).

Assess changes to mitochondrial function and mitophagy pathways following radiation treatment of TRAMPC1 cultures and effects of p62-mitophagy inducer versus cinaciguat. It has been demonstrated that mitochondrial dysfunction is one of the earliest events following exposure to ionizing irradiation with effects lasting months. Therefore, the radiation-mediated downregulation of mitochondrial respiration may be characterized using cultured TRAMP-C1 cells at different time points after radiation exposure. TRAMP-C1 cells may be cultured in T75 flasks to ˜80% confluence and exposed to 5 fractions of 6 Gy irradiation. Cells may be harvested at 1, 3 and 7 days after irradiation, lysed using a bead homogenizer (FastPrep-24, MP Biomedical) and subjected to differential centrifugation to isolate mitochondria for RCR measurements. Separate sets of cultures may be treated with cinaciguat (1 μM) or p62-mitophagy inducer (10 μM) during irradiation.

Separate cultures may be made on 100 mm² dishes for protein and mRNA isolation and subjected to the irradiation/drug treatment protocol used for RCR experiments above. Protein and mRNA levels of parkin/PINK1 (ubiquitin-mediated mitophagy pathway), p62 (adaptor protein in transport of ubiquitinated mitochondria), HIF-1α (hypoxia induced factor-1a; a marker for mitophagy), pAMPK (5′-AMP-activated protein kinase, inducer of parkin-dependent and independent mitophagy) may be evaluated under the different treatment conditions.

Determine the differential effect of p62-mitophagy inducer versus cinaciguat on cell senescence and reemergence following radiation treatment of orthotopic luciferase-expressing TRAMP-C1 tumors. In this Example, one of the most important unanswered questions in treating senescence tumor cells to prevent their reemergence may be addressed: whether to promote mitophagy and recover the cells, or apoptosis and clear them. This is relevant as mitochondria can exploit the proapoptotic effects of certain drugs while being responsible for the development of resistance with others, and thus the answer may be drug dependent. Alternatively, it may depend upon the level of oxidative stress, with moderate levels leading to mitophagy and tumor progression, while high levels lead to apoptosis and inhibition of reemergence. However, preliminary data suggest that the therapeutic benefits of cinaciguat in treating orthotopic tumors, after fractionated EBRT, involve increases in cGMP levels, and decreases in NF-κβ expression (senomorphic effect) and Bcl-2/BAX ratios (senolytic effect).

Accordingly, two weeks after the last of five daily 6 Gy fractions, non-irradiated and irradiated mice with/without prostate tumors, may be treated with p62 mitophagy inducer (0.1-1 mg/kg/day), cinaciguat (0.1-1 mg/kg/day) or vehicle for 28 days using subcutaneous implanted osmotic pumps. Mice may be examined non-invasively during this treatment period using bioluminescence imaging and urine spot tests after which they may be sacrificed and prostate tumor tissues collected for mitochondrial respiration and molecular assessments. RCRs may be calculated based on state III/IV respiration rates as shown in FIG. 8 and detailed in the methods. As with in vitro cells studies, above, protein and mRNA levels of parkin/PINK1, p62, HIF-1α, and pAMPK may be evaluated for the different experimental paradigms. In addition, p62 and pAMPK may also be evaluated for intensity and area of positive staining in paraffin embedded tissue section using immunofluorescence or chromogen staining.

When parkin binds to PINK1 on the inner mitochondrial membrane, it promotes mitophagy, so it is anticipated that p62 mitophagy inducer will allow recovery from radiation induced senescence to promote tumorigenesis. AMPK is a regulator of cellular homeostasis that helps cells adapt to energetic stresses where its activation promotes ATP-generating catabolic processes while simultaneously inhibiting ATP-depleting anabolic ones. Thus, elevated expression usually indicates oxidative stress and the onset or pending senescence. However, there are distinct subcellular locations of AMPK subunits whose activation could promote tumor suppressive or oncogenesis. Thus, changes in AMPK expression should be used to support more definitive markers such as decreases in the Bcl-2/BAX ratio for apoptosis and an increase in HIF-1α expression for mitophagy.

It is anticipated that in vitro experiments will show irradiation decreases mitochondrial respiration after a single dose with progressive decreases in activity with increasing number of fractions. Preliminary data indicate p62-mitophagy inducer promotes tumor survival and growth after irradiation. If the drug is indeed acting on the mitophagy pathways, then it is anticipated that p62 treatment will restore RCR to pre-irradiation level by reducing the number of dysfunctional mitochondria and recovering senescence tumor cells. Cinaciguat is expected to promote apoptosis of senescent cells thereby clearing the TRAMP-C1 cells. With orthotopic tumors, it is anticipated that cinaciguat may increase mitophagy to recover the cells risking reemergence, however, concurrent apoptosis will likely clear them instead.

### Example 3

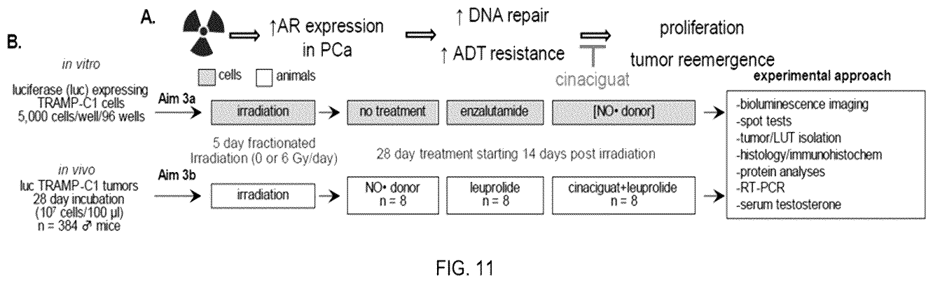

Determine the effects of a nitric oxide donor versus androgen signaling inhibition following radiation treatment on cell senescence and tumor reemergence. Radiation treatment of localized prostate tumors is almost always accompanied by ADT to inhibit growth and survival of irradiated tumor cells. However, 10-20% of patients will develop castration-resistant prostate cancer (CRPC) by around 5 years of follow up. These patients can be further treated with androgen blockers (e.g., enzalutamide) and other chemotherapeutics; however, there are limited options for AR-independent, CRPC with an overall poor prognosis. Thus, the interaction of cinaciguat with the AR signaling using the TRAMP-C1 model is examined following irradiation. Along with the possibility of sGC modulation as a supplement or replacement for current AR targeted therapies. Effects on downstream elements of the AR pathway is addressed in cultured cells and the effects of radiation combined with ADT may be examined with orthotopic TRAMP-C1 tumors (FIG. 11).

Determine the effects of a NO· donor and androgen receptor blocker on radiation induced cell senescence in TRAMP-C1 cultures. Preliminary studies demonstrated single dose 8 Gy irradiation could induce cell death which was potentiated by cinaciguat but not enzalutamide or DETA NoNoate (FIG. 4 (A)). It is unclear whether these agents alter development of cell senescence which have implications for tumor recurrence. To address this, fractionated irradiation (6 Gy/day/5 days) may be performed on cultured luc-TRAMP-C1 cells in 96 well plates in the presence of different concentrations of the AR blocker, enzalutamide (0.01-10 μM), long-acting NO donor, DETA NoNoate (0.1-100 μM) or vehicle (DMSO). Cultures may be examined by bioluminescence to track cell survival and after one week may be examined for β-galactosidase activity (Thermofisher, #75707). Based on 96-well plate studies, western blot and RT-PCR studies may be performed using the most efficacious drug concentrations. For protein and RNA collection, TRAMP-C1 cells may be plated in 100 mm² culture dishes (˜1×10⁶ cells), subjected to fractionated irradiation and treated with enzalutamide or DETA-NoNoate for a one-week period starting four days after the final irradiation dose. Cells may be harvested according to standard protocols for protein or RNA collection. Protein levels of AR, sGC subunits, NOS subtypes, p21/p16 (senescence markers), caspase-3/9 (apoptosis markers), IL-1β/I-6 (tumorigenic inflammatory cytokines) may be examined by western blot or ELISA. The mRNA expression of AR splice variants, which are associated with development of CRPC, NOS subtypes, sGC subunits may be evaluated using qRT-PCR.

Assess differential effect of post-irradiation treatment with NO· donor versus leuprolide-induced androgen ablation on cell senescence and reemergence of orthotopic TRAMP-C1 tumors. ADT is a first-line treatment for prostate cancer in conjunction with radiotherapy and/or surgery. However, ADT itself promotes cellular senescence which in turn can induce androgen resistance. Therefore, irradiation together with ADT likely compounds the accumulation of SASP. To examine this possibility, orthotopic luc-TRAMP-C1 cell may be injected in mice that are subjected to fractionated irradiation protocol as outlined in the previous aims. At 14 days after the final irradiation, mice may be separated into three cohorts. The first group may receive daily intraperitoneal injections of nitroglycerine (1 mg/kg/day) for 28 days. The second group may receive a single intraperitoneal injection of leuprolide (0.5 mg total). The last cohort may receive a single dose of leuprolide and may be implanted with a subcutaneous osmotic pump containing cinaciguat (1 mg/kg/day/28 days). During treatment, tumor size may be monitored weekly by bioluminescence imaging, two-hour urine spot tests and tail vein blood collection may be performed for serum testosterone measurements. Mice may be monitored for an additional 28 days after completion of drug treatment to determine if there is tumor regrowth. At the experimental endpoint, mice may be sacrificed for tissue collection.

Prostate tissues may be processed for histological, protein analysis and RT-PCR studies. Tissue may be histologically examined for β-galactosidase activity and immunolabeled for AR, NF-κB, p21/p16, Ki67 and Bcl-2 in frozen sections. Sections may be imaged and quantified based on the number of labeled cells per total number of nuclei in the imaged area. Protein levels of sGC subunits, NOS subtypes, AR, caspases-3/9 and SASP associated cytokines may be evaluated using western blot or ELISA as appropriate. The expression of AR splice variants, sGC subunits and NOS subtypes may also be quantified by qRT-PCR from tissue samples.

Based on reported data where NO· donors decrease AR expression in prostate cancer it is hypothesized that cinaciguat will decrease AR expression and downstream effectors following irradiation to a greater extent than DETA-NoNoate, due to higher affinity of cinaciguat to oxidized sGC. It is anticipated from in vivo studies, that irradiation combined with ADT will have greater number of senescent cells compared to individual treatments which could potentially increase the risk of tumor regrowth. It is predicted that cinaciguat treatment with ADT will not be more effective than cinaciguat alone in reducing accumulation of senescent cells, associated inflammation and subsequent tumor regrowth.

### Materials and Methods—Examples 1-3

Cell culture. Luciferase expressing TRAMP-C1 cells may be maintained in DMEM supplemented with 10% FBS, 1% PenStrep, 5 μg/ml insulin and 10 nM dehydroisoandrosterone and used at 10-15 passages. For experiments in Examples 1-3 cells may be plated at 5,000 cells/well in 96-well plates.

Tumor implantation. Mice may be anesthetized with isoflurane (5% initiation-1.5% maintenance) and following depilation, injected with 5×10⁶ cells/50 μl into ventrolateral prostatic lobes bilaterally through a small midline abdominal incision. The incision may be suture closed in two layers and tumors allowed to grow for 4-6 weeks.

Bioluminescence in vitro and in vivo imaging. Cells may be treated with 150 μM luciferin D and immediately placed in Tecan SPECTRA Fluor Plus plate reader for bioluminescence measurement. Readings may be averaged for each group of cells (n≥ 8) and background (empty well readings) subtracted. Mice may be anesthetized with avertin (300 mg/kg, IP), depilated and injected with 150 mg/kg luciferin D, IP. They may be anesthetized in a BioRad ChemiDoc MP imaging system in the supine position. Bioluminescence may be imaged for 600 sec using the Chemi Hi sensitivity protocol, intensity expressed colorimetrically, superimposed over images of the mice.

Blood pressure (BP) and heart rate (HR) photoplethysmography. Mean arterial BP and HR may be measured weekly in mice during infusion pump administration cinaciguat using non-invasive tail cuff measurements (BP-2000, Visitech Inc.).

Selective prostate and 96 well plate irradiation. Mice may be anesthetized with avertin (300 mg/kg, IP), depilated and visualization via bioluminescence, placed in X-RAD320 irradiator in the supine position. The radiation beam (320kVpeak) may be collimated to ensure that only the tumors receive fractionated irradiation (6 Gy×5 days). The 96 well plates may have the collimator set to selectively irradiate one or more rows of cells.

Mitochondrial respiration. Mice may be anesthetized with isoflurane, prostates w/wo tumors excised and placed in cold mitochondrial solution (MS, mM): HEPES, 5.0; KCl, 125; KH2PO4, 2.0; EDTA, 0.02; MgCl2, 5.0; BSA, 0.2 mg/ml; pH 7.4 with 100 mM KOH. Tissue may be sectioned (10 μm) using a Mcllwain tissue chopper (Brinkmann), placed in MS (10 ml) and homogenized with a Teflon pestle (75 rpm). The homogenate may be spun (1,000×g; 10 min) and the supernatant re-spun (10,000×g; 10 min) to obtain a pellet containing mitochondria. Pellet may be resuspended in MS (100 μl) and 25-μl placed in a gas-tight vessel containing a Clark O2 microelectrode (MI-730/OM-4; Microelectrodes, NH) to measure state 3 (succinate+ADP) and state 4 (succinate alone) respiratory rates. Electrode may be calibrated against dissolved O₂ in MS after air equilibration at 36° C. (215 μM) and zeroed with sodium dithionite. The RCR, measures “tightness of coupling” between electron transport and oxidative phosphorylation, the ratio of state 3 to state 4. An RCR of 2-4 may be good for complex II substrate.

Tissue preparation. At the end of in vivo experiments animals may be humanely euthanized and tissues dissected for histology and molecular examination.

DNA measurements. Qiagen DNeasy blood and tissue kit may be used to isolate DNA from left lateral and ventral prostatic lobes and the concentrations measured by absorbance A260/A280 (NanoDrop, Thermo Fisher).

DNA measurements. Qiagen DNeasy blood and tissue kit may be used to isolate DNA from left lateral and ventral prostatic lobes and the concentrations measured by absorbance A260/A280 (NanoDrop, Thermo Fisher).

Histology/immunohistochemistry. Tissues may be isolated, fixed overnight in 10% buffered formalin, embedded in paraffin, and sectioned 3 μm thick on a microtome. Sections may be stained with H&E, Verhoeff van Gieson, or immunohistochemical/fluorescence and imaged using confocal microscopy. For immunohistochemistry, tissues may be frozen in OCT, fixed in 4% buffered formalin and sectioned 7-10 μm thick on a cryostat.

Metabolic cage measurements of voiding function. Animals may be placed unrestrained in metabolic cages where load cells (5 μl/10 Hz sensitivity) below each animal may record duration and volume of voids, and filter paper the voiding patterns; data may be recorded up to 48 hrs with light/dark cycles using LabChart and OxyMax software.

Western blot, ELISA and RT-qPCR. Tissue samples may be collected immediately following sacrifice of the animals and snap frozen. Plasma, tissue and protein lysates and total RNA may be isolated and used for western blot, ELISA and RT-qPCR analysis accordingly to manufacturer's protocols or as previously described.

Statistical analysis. Data from experiments and image analysis may be summarized as meantstandard deviation. ANOVA with interaction terms may be performed to determine between group differences followed by Fisher's LSD post-hoc tests to determine differences between specific group-treatment-time point combinations of interest. The null hypotheses may be rejected when p<0.05.

Sample size justification. Prior data from experiments, achieving 80% statistical power in two-tailed tests conducted at the α=0.05 level, and published statistical methods implemented in commercially available statistical software (e.g., PASS 2021®, Number Cruncher Statistical Systems, Kaysville, Utah) may be used for computations. From prior experiments, it may be conservatively estimated between-animal standard deviations for serum testosterone may be 50 pg/ml and mitochondrial respiration RCR may be 0.9. Moreover, based on observed ranges, standard deviation for bioluminescence imaging diameter may be conservatively inferred as 3.75 mm². With 8 animals per group/condition/measurement combination, differences as small as 27.1 pg/ml in serum testosterone, 1.4 in mitochondrial respiration RCR, and 5.65 in bioluminescence imaging diameter with 80% statistical power may be detected in two-tailed tests conducted at the α=0.05 level.

### Example 4—Cystitis and Fibrosis

Late radiation cystitis is an adverse event associated with irradiation of pelvic tumors (including prostate tumor) that occurs months to years after the completion of therapy. Symptomatic radiation cystitis appears in 5-10% of cases, presents with pain, incontinence and recurrent hematuria, may be life-threatening and there are currently no non-invasive or effective treatments. Although pathophysiology of late cystitis remains largely unclear, urothelial as well as endothelial cells play an important role. These cells are among the most sensitive to radiation insult and their obliteration leads to inflammation, hypoxia and, ultimately, bladder fibrosis. Since cellular senescence may be a stress response due to DNA damage following irradiation, urothelial senescence can be a contributing factor to the emergence of late radiation cystitis. P21 is a cyclin-dependent kinase inhibitor that maintains the viability of DNA damage-induced senescent cells and is a reliable senescence marker. Nuclear localization of p21 expression in control and irradiated bladders was analyzed and demonstrated increased expression of p21 in irradiated bladders, especially in the urothelial layer, with further significant increase at a later stage. Thus, cellular senescence is involved in the urothelial response to radiation insult, is exacerbated over time and may play an important role in the emergence of late radiation cystitis. The beneficial effect of cinaciguat in preventing/reversing bladder fibrosis and dysfunction may involve the senolytic removal of senescent cells.

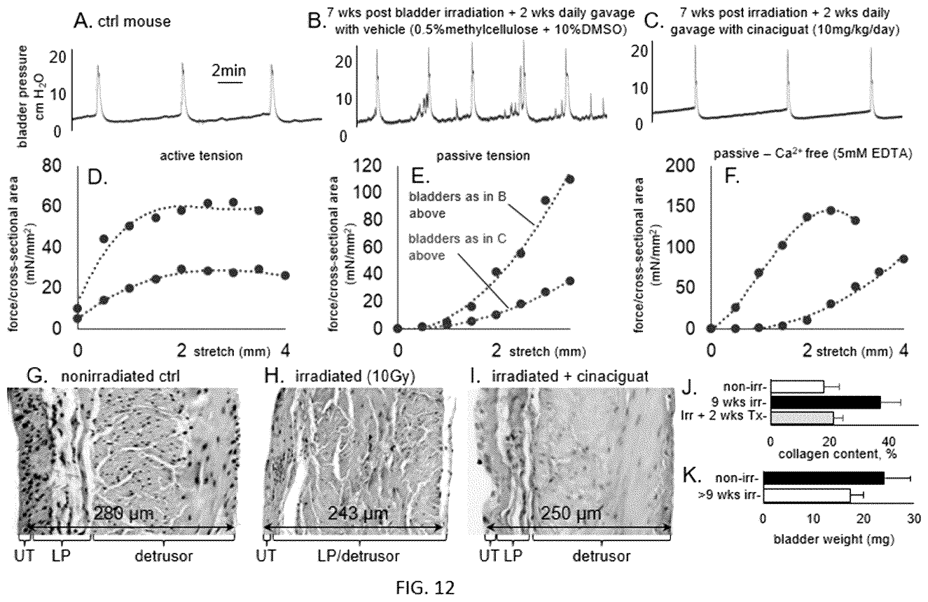

Decerebrate CMG in mice with chronic radiation cystitis (>8 weeks post exposure) showed a significant decrease in intercontractile intervals (ICIs), non-voiding contractions, and decreased compliance (FIG. 12 (B) vs. FIG. 12 (A)). Daily gavage of irradiated mice with cinaciguat (10 mg/kg/day) starting 7 weeks post irradiation, normalized the CMG profile (FIG. 12 (C) vs. FIG. 12 (B)). Length-tension measurements from isolated bladder strips showed a significant decline in active force generation (FIG. 12 (D)) and increased passive tension/tissue stiffness (FIG. 12 (E, F)) 9 weeks following irradiation. Staining for collagen showed significant deposition in chronically irradiated mice compared to controls (FIG. 12 (H) vs. FIG. 12 (G)). Chronically irradiated mice treated with cinaciguat showed a bladder wall structure similar to non-irradiated controls (FIG. 12 (I)), supporting enhanced NO·-mediated signaling reverses radiation-induced fibrosis. FIG. 12 (J) demonstrates doubling of collagen content 9 weeks post irradiation that was reversed by cinaciguat. Furthermore, irradiation decreases bladder weight by approximately 30% that could be attributed to muscle atrophy (FIG. 12 (K)).

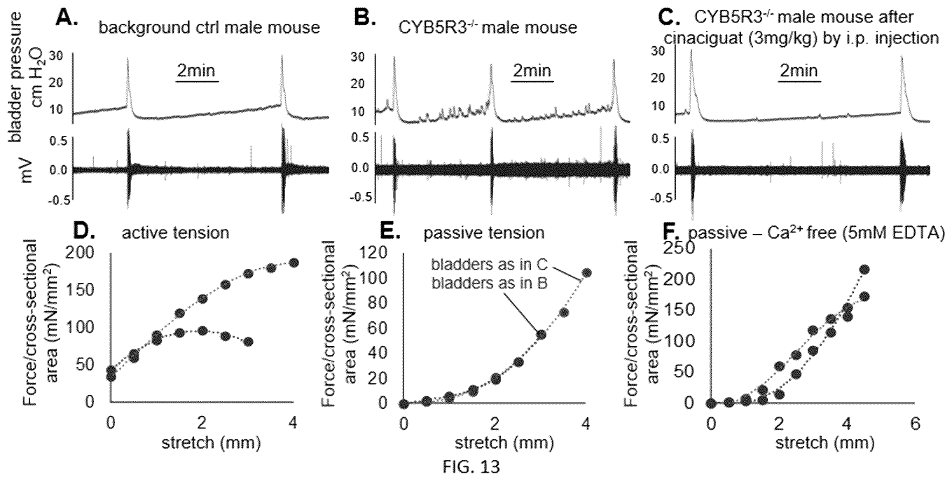

CyB5R3 smooth muscle conditional knockout mice showed non-voiding contractions and shortened ICI compared to controls (FIG. 13 (B) vs. FIG. 13 (A)). Intraperitoneal injection of cinaciguat increased ICI and eliminated non-voiding contractions (FIG. 13 (C)). Thus, impaired sGC signaling adversely affects bladder function. Length-tension measurement from CyB5R3^{-/-} mice showed a decrease in force generation (FIG. 13 (D)) but no significant change in bladder tissue compliance at 10 days after CyB5R3 deletion by systemic tamoxifen administration (FIG. 13 (E-F)) (Zabbarova IV et al. J Pathol. 2022 April; 256 (4): 442-454).

Immunohistochemical staining for p21 was performed in non-irradiated and irradiated mouse bladders 2 weeks and 9 months following irradiation.

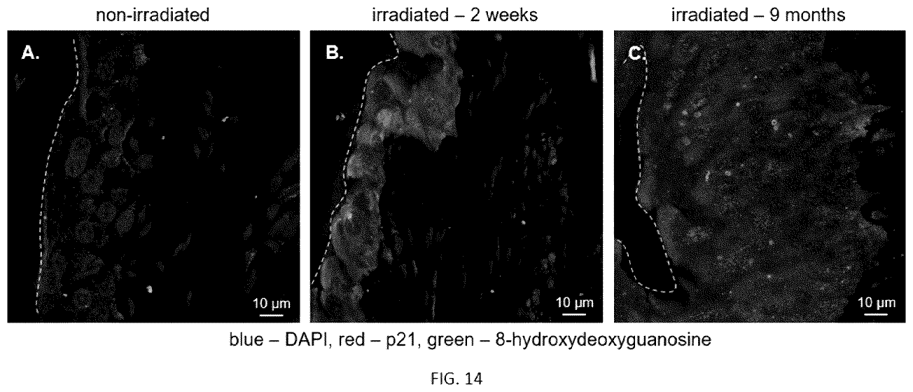

Immunohistochemical staining of the tissues demonstrated increased expression of p21 in irradiated bladders, especially in urothelial layer, with further significant increase at a later stage (FIG. 14). There was also a concurrent increase in labeling for 8-hydroxydeoxyguanosine (green) which is a marker of oxidative DNA damage. The number of p21-positive urothelial cells in age-matched control tissues was 97±20 cells/mm², two weeks post irradiation, 347±57 cells/mm², and nine months later, 834±315 cells/mm².

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

The invention claimed is:

1. A method of treating a patient having a cancer, comprising administering to the patient after initiation of a primary cancer therapy in the patient an amount of a soluble guanylate cyclase (sGC) activator effective to promote senolytic activity and/or promote senomorphic activity in the patient.

2. The method of claim 1, wherein the patient has prostate cancer or has been treated for prostate cancer using the primary cancer therapy.

3. The method of claim 1, wherein the primary cancer therapy is radiation therapy or chemotherapy.

4. The method of claim 1, in which cells of the patient, such as cancer cells of the patient, exhibit a senescence-associate secretory phenotype (SASP).

5. The method of claim 5, wherein the senescent cells exhibiting a senescence-associate secretory phenotype (SASP) are cancer cells, e.g., prostate cancer cells.

6. The method of claim 1, wherein the sGC activator is cinaciguat.

7. The method of claim 1, wherein the sGC activator is:
a. a compound of formula I: 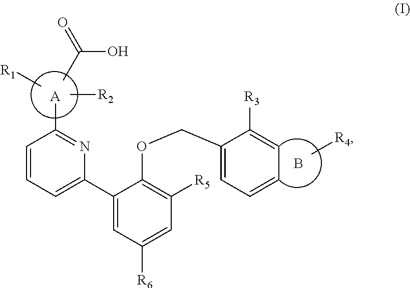
wherein:
A is a 5- or 6-membered aryl, heteroaryl or heterocyclyl group;
B is a 5-7 membered heterocyclyl group containing one nitrogen, wherein one carbon of the heterocyclyl group is optionally substituted with an oxo group, or B is a 5-membered heteroaryl group containing at least 2 nitrogens;
R₁ and R₂ are independently selected from H, C₁₋₄alkyl, C₃₋₆cycloalkyl, tetrahydropyranyl, —CF₃, —CH₂CF₃ and —CH₂CH₂CO₂H;
R₃ is selected from H and —CH₃;
R₄ is selected from H, C₁₋₆alkyl, C₃₋₇cycloalkyl, —C(O)C₁₋₆alkyl, —CH₂CF₃, —SO₂C₁₋₆alkyl, —SO₂(CH₂)₁₋₃CO₂H, —CO₂C₁₋₄alkyl, heterocyclyl, aryl, heteroaryl,
(C₀₋₂-alkyl) heterocyclyl, —(C₁₋₂alkyl) aryl and —(C₁₋₂alkyl) heteroaryl,
wherein said heterocyclyl is selected from tetrahydrofuranyl, azetidinyl, pyrrolidinyl, pyranyl, tetrahydropyranyl, dioxanyl, thiomorpholinyl, 1,1-dioxo-1λ⁶-thiomorpholinyl, morpholinyl, piperidinyl, piperazinyl, and azepinyl, and
said heterocyclyl, cycloalkyl, aryl and heteroaryl are optionally substituted with one to two groups independently selected from C₁₋₃alkyl, —CF₃, and halogen,
or R₄ is optionally not present when B is a heteroaryl group;
R₅ is selected from H, —C₁₋₃alkyl, —OCH₃, —CF₃, —CN and Cl; and R₆ is selected from H and CH₃, provided that R₅ and R₆ are not both H;
or a salt thereof; or
b. is selected from one or more of: 5-chloro-2-[(5-chlorothiophen-2-yl)sulfonylamino]-N-(4-morpholin-4-ylsulfonylphenyl)benzamide); 5-cyclopropyl-2-[1-[(2-fluorophenyl)methyl]pyrazolo[3,4-b]pyridin-3-yl] pyrimidin-4-amine; (3S)-3-(4-chloro-3-{[(2S,3R)-2-(4-chlorophenyl)-4,4,4-trifluoro-3-methylbutanoyl]amino}phenyl)-3-cyclo-propylpropanoic acid; 5-{(4-carboxybutyl) [2-(2-{[3-chloro-4′-(trifluoromethyl) biphenyl-4-yl]methoxy}phenyl)ethyl]amino}-5,6,7,8-tetrahydroquinoline-2-carboxylic acid; and 5-{[2-(4-carboxy-phenyl)ethyl][2-(2-{[3-chloro-4′-(trifluoromethyl) biphenyl-4-yl]methoxy}phenyl)ethyl] amino}-5,6,7,8-tetrahydroquinoline-2-carboxylic acid, or a salt thereof.

8. A method of clearing senescent cells and/or increasing senomorphic activity in a patient comprising administering to the patient an amount of a soluble guanylate cyclase (sGC) activator effective to clear senescent cells and/or increase senomorphic activity in the patient.

9. The method of claim 8, wherein a primary cancer therapy is administered or has been administered to the patient prior to administering the soluble guanylate cyclase (sGC) activator to the patient.

10. The method of claim 9, wherein the patient has prostate cancer or has been treated for prostate cancer using the primary cancer therapy.

11. The method of claim 9, wherein the primary cancer therapy is radiation therapy or chemotherapy.

12. The method of claim 8, in which cells of the patient, such as cancer cells of the patient, exhibit a senescence-associate secretory phenotype (SASP).

13. The method of claim 12, wherein the senescent cells exhibiting a senescence-associate secretory phenotype (SASP) are cancer cells, e.g., prostate cancer cells.

14. The method of claim 8, wherein the sGC activator is:
a. cinaciguat;
b. a compound of formula I: 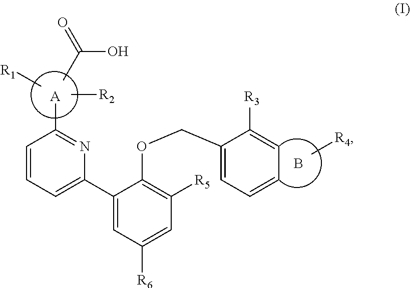
wherein:
A is a 5- or 6-membered aryl, heteroaryl or heterocyclyl group;
B is a 5-7 membered heterocyclyl group containing one nitrogen, wherein one carbon of the heterocyclyl group is optionally substituted with an oxo group, or B is a 5-membered heteroaryl group containing at least 2 nitrogens;
R₁ and R₂ are independently selected from H, C₁₋₄alkyl, C₃₋₆cycloalkyl, tetrahydropyranyl, —CF₃, —CH₂CF₃ and —CH₂CH₂CO₂H;
R₃ is selected from H and —CH₃;
R₄ is selected from H, C₁₋₆alkyl, C₃₋₇cycloalkyl, —C(O)C₁₋₆alkyl, —CH₂CF₃, —SO₂C₁₋₆alkyl, —SO₂(CH₂)₁₋₃CO₂H, —CO₂C₁₋₄alkyl, heterocyclyl, aryl, heteroaryl,
(C₀₋₂alkyl) heterocyclyl, —(C₁₋₂alkyl) aryl and —(C₁₋₂alkyl) heteroaryl,
wherein said heterocyclyl is selected from tetrahydrofuranyl, azetidinyl, pyrrolidinyl, pyranyl, tetrahydropyranyl, dioxanyl, thiomorpholinyl, 1,1-dioxo-1λ⁶-thiomorpholinyl, morpholinyl, piperidinyl, piperazinyl, and azepinyl, and
said heterocyclyl, cycloalkyl, aryl and heteroaryl are optionally substituted with one to two groups independently selected from C₁₋₃alkyl, —CF₃, and halogen,
or R₄ is optionally not present when B is a heteroaryl group;
R₅ is selected from H, —C₁₋₃alkyl, —OCH₃, —CF₃, —CN and Cl; and R₆ is selected from H and CH₃, provided that R₅ and R₆ are not both H;
or a salt thereof; or
c. is selected from one or more of: 5-chloro-2-[(5-chlorothiophen-2-yl)sulfonylamino]-N-(4-morpholin-4-ylsulfonylphenyl)benzamide); 5-cyclopropyl-2-[1-[(2-fluorophenyl)methyl]pyrazolo[3,4-b]pyridin-3-yl] pyrimidin-4-amine; (3S)-3-(4-chloro-3-{[(2S,3R)-2-(4-chlorophenyl)-4,4,4-trifluoro-3-methylbutanoyl]amino}phenyl)-3-cyclo-propylpropanoic acid; 5-{(4-carboxybutyl)[2-(2-{[3-chloro-4′-(trifluoromethyl)biphenyl-4-yl]methoxy}phenyl)ethyl]amino}-5,6,7,8-tetrahydroquinoline-2-carboxylic acid; and 5-{[2-(4-carboxy-phenyl)ethyl][2-(2-{[3-chloro-4′-(trifluoromethyl) biphenyl-4-yl]methoxy}phenyl)ethyl] amino}-5,6,7,8-tetrahydroquinoline-2-carboxylic acid, or a salt thereof.

15. The method of claim 9, wherein the patient's bladder is exposed to the primary cancer therapy, and the soluble guanylate cyclase (sGC) activator is administered after the primary therapy.

16. The method of claim 15, wherein the primary cancer therapy is radiation therapy, and the patient's bladder is exposed to the radiation therapy.

17. The method of claim 15, wherein the primary cancer therapy is administered to treat prostate cancer in the patient.

18. The method of claim 9, wherein the patient has noninfectious cystitis after a primary cancer therapy.

19. The method of claim 9, wherein the soluble guanylate cyclase (sGC) activator is administered after the primary cancer therapy to treat fibrosis in the patient.

20. The method of claim 8, for treatment of fibrosis in the patient.
